(19) United States
(12) Patent Application Publication
(10) Pub. No.: US 2008/0311184 A1
(43) Pub. Date: Dec. 18, 2008

(54) METHOD FOR DEVELOPING, TESTING, AND USING ASSOCIATES OF MACROMOLECULES AND COMPLEX AGGREGATES FOR IMPROVED PAYLOAD AND CONTROLLABLE DE/ASSOCIATION RATES

(75) Inventor: Gregor Cevc, Kirchheim (DE)

Correspondence Address: WILSON SONSINI GOODRICH & ROSATI, 650 PAGE MILL ROAD, PALO ALTO, CA, 94304-1050, US

(73) Assignee: IDEA AG, MUNCHEN (DE)

(21) Appl. No.: 11/929,544

(22) Filed: Oct. 30, 2007

Related U.S. Application Data
(63) Continuation of application No. 09/555,986, filed on Aug. 17, 2000, now abandoned.

(51) Int. Cl. A61K 9/127; A61K 38/00; A61K 45/00; A61K 38/21; A61K 39/395; A61K 31/7088; A61K 31/70; A61K 38/43; A61P 43/00

(52) U.S. Cl. 424/450; 514/3; 424/85.2; 424/85.5; 424/85.6; 424/85.7; 424/178.1; 514/12; 514/44; 514/54; 424/94.1

(57) ABSTRACT

This invention describes the principles and procedures suitable for developing, testing, manufacturing, and using combinations of various amphipatic, if necessary modified, macromolecules (such as polypeptides, proteins, etc.) or other chain molecules (such as suitable, e.g. partly hydrophobised, polynucleotides or polysaccharides) with the aggregates which comprise a mixture of polar and/or charged amphipats and form extended surfaces that can be freely suspended or supported. The described methods can be utilised for the optimisation of aggregates that, after association with chain molecules exerting some activity or a useful function, are suitable for the application in vitro or in vivo, for example, in the fields of drug delivery, diagnostics or bio/catalysis. As special examples, mixtures of vesicular droplets consisting of lipids loaded (associated) with insulin, interferon, interleukin, nerve growth factor, calcitonin, and an immunoglobulin, etc., are described.

This application is a Continuation of U.S. patent application Ser. No. 09/555,986, filed Aug. 17, 2000, which is the U.S. National Stage of PCT International Patent Application No. PCT/EP98/06750, filed Oct. 23, 1998, each of which is incorporated by reference herein in its entirety.

The invention concerns combinations of substances which exhibit amphipatic properties and can form extended surfaces, especially membrane-like surfaces, when in contact with a liquid medium. More specifically, the invention concerns the association of other amphipatic substances, on a molecular level, with such surfaces, whereby such other amphipatic, surface-associating substances are typically larger molecules with repeating subunits such as oligomers and polymers, and often stem from the class of biologically active agents.

The invention further concerns methods of making such surfaces and of producing associates between such larger molecules and surfaces as well as various uses of such surfaces and associates.

Amphipatic chain molecules and related macromolecules, such as proteins, absorb to any kind of surface but not to the same amount and, most often, in a different conformation. This invention describes the state of the art and provides a new rationale for optimizing and controlling the macro-molecular association with soft, complex surfaces. This should be valuable for future biological, biotechnological, pharmaceutical, therapeutic, and diagnostic applications.

(Macro)molecular absorption/binding to an adsorbent surface (adsorbent/adsorbate association) is a multi-step process:

i) the hi step includes adsorbate redistribution, preferably accumulation, at the absorbent/solution interface. This step is typically fast and diffusion-rate controlled.
ii) in the second step, adsorbate molecules hydrophobically associate with the soft (membrane) surface. The process involves several stages, such as partial molecular binding and sequential rearrangement(s), at least some of them often being slow.

It has been argued (Cevc, G., Strohmaier, L., Berkholz, J., Blume, G. Stud. Biophys. 1990, 138: 57ff) that the probability for a large molecule to bind specifically to a surface-attached ligand embedded into a “soft” lipid membrane is diminished by the proximity of an interface. This appears to be due to the same non-Coulombic, hydration-dependent force which also prevents the colloidal collapse of adjacent lipid membranes onto each other. Total resulting force decreases with decreasing hydrophilicity and stiffness of the lipid-solution interface (Cevc, G., Hauser, M., Kornyshev, A. A. Langmuir 1995, 11: 3103-3110).

It has also been previously conjectured that the extent of non-specific protein adsorption to a lipid bilayer (Cevc, et al., op. cit.: 1990) is proportional to the availability of hydrophobic binding sites for the protein in a membrane. Creating the defects in the lipid bilayers mechanically (e.g. by sonication) or by inducing lipid phase transitions was found to increase the amount of membrane-bound protein.

It is generally believed that the more hydrophobic the surface, the greater is the extent of amphipatic macromolecules' adsorption. For example, K. Prime and G. M. Whitesides (Science, 1991, 252: 1164-1167), who used self-assembled monolayers of long chain alkanes with terminal groups of differing hydrophobicity to systematically vary the adsorption of proteins via hydrophobic amino acids binding, confirmed this “rule” or “principle”. To date, “hydrophobic attraction” is therefore considered to be the dominant force in protein adsorption.

On the other hand, it is widely accepted that the net macroscopic interaction between a hydrophilic macromolecule, such as a protein, and a hydrophilic surface, such as glass or montmorillonite clay, immersed in an aqueous solution at neutral pH is dominated by strong repulsion. Thus, under conditions where the macroscopic-scale rules of van der Waals, Lewis acid-base, and electrical double layer interactions are applicable, adsorption of hydrophilic proteins onto hydrophilic mineral surfaces is normally weak (H. Quiquampoix et al, Mechanisms and Consequences of Protein Adsorbtion on Soil Mineral Surfaces, Chapter 23 in Proteins at Interfaces (PAI), T. A. Horbett and J. L. Brash, eds., ACS Symposium Series 602, 1995, New York 321-333). Some hydrophilic proteins do adsorb onto glass from a solution, however, albeit more sparsely than they would adsorb onto a hydrophobic surface; such proteins also adsorb onto montmorillonite clay surfaces. To explain this non-trivial phenomenon it was proposed, and supported by experimental data, that proteins can bind to an equally (e.g. negatively) charged hydrophilic mineral surface, immersed in an aqueous medium, via plurivalent counterion (e.g. calcium) binding to the (negatively) charged hydrophilic proteins. Other subtle charge effects involve the formation of hydrogen bonds, salting-in of proteins, and the binding of counterions. For example, it was suggested that “structural rearrangements in the protein molecule, dehydration of the sorbent surface, redistribution of charged groups and protein surface polarity” may all affect protein adsorption (Haynes, C. A. et. Al, Colloids Surface B: Biointerfaces, 2, 1994: 517-566). In agreement with this, Coulombic interactions, although important, in general do not dominate protein adsorption to solid surfaces, as it is the case of strong adsorption of α-LA (alpha-lactalbumin) to PS (polystyrene) at conditions where the protein carries a substantial net negative charge. Another recent survey conceded that “no clear consensus has developed to date as to the extent of charge effect on protein adsorption” (Reversibility and the Mechanism of Protein Adsorption, W. Norde and C. Haynes, Chapter 2 in (PAI), op. cit.: 26-40).

For soft surfaces, such as membranes, the view currently prevails that at least the first steps in protein adsorption are electrostatics-driven and/or charge dominated (see, for example: Deber, C. M.; Hughes, D. W.; Frasez, P. E.; Pawagi, A. B.; Moscarello, M. A. Arch. Biochem. Biophys. 1986, 245: 455-463; Zimmerman, R. M., Schmidt, C. F., Gaub, N. H. E. J. Colloid Int. Sci. 1990, 139: 268-280; Hernandez-Caseldis, T., Villalaain, J.; Gomez-Fernandez, J. C. Mol. Cell. Biochem. 1993, 120: 119-126.). Leading experts have also concluded that electrostatic forces are critical for the binding of the secretory phospholipases to various lipid aggregates (Scott, D. L.; Mandel, A. M.; Sigler, P. B.; Honig. B. Biophys. J. 1994, 67: 493-504).

Until now, skilled people believed that the chief determinant of final protein adsorption is the hydrophobic attraction, while the ionic interactions, combined with entropy gain caused by conformational changes of the protein during its adsorption, also play some role.

Proteins typically adsorb strongly to oppositely charged surfaces, but not to surfaces that bear equal charges. pH dependence of protein adsorption reflects this fact. The charge effects can sometimes be confounded by “lurking” factors, such as small multivalent counterions, which can bridge protein and surface sites with a similar charge, which would normally be expected to repel each other.

The final conformation, of an adsorbed protein is seldom identical to the starting conformation. This is the reason why most models of protein adsorption invoke a transition from a reversibly adsorbed state to a more tightly held state, which arises in consequence to a molecular restructuring or relaxation of the protein on the surface. Macromolecular rearrangement upon adsorption is often catastrophic and culminates in protein denaturation. From the fact that enzymes and antibodies retain at least some of their biological activity in the adsorbed state, and biologic activity is exquisitely dependent on maintenance of a native structure, it can be concluded, however, that changes in adsorbed proteins conformation are often limited in time and scope. Protein folding is most strongly affected by hydrophobic interactions. Both phenomena, protein binding and conformation changes, are sensitive to the presence of certain amphiphiles, such as surfactants and phospholipids. Protein adsorption was believed to decrease, or be reversed by the addition of such molecules.

Proteins are therefore, more often than not, mixed with surfactants during protein isolation, in order to minimise non-specific protein adsorption and loss. In one particular study, the adsorption of proteins decreased to a negligible level as the surface concentration of grafted Pluronic surfactant increased. The number of ethylene-glycol (EG) units in the monomer side-chain of surfactant was 4, 9, and 24, the monomer with the smallest number of EG units (4) being the most “inert” toward the blood components (Analysis of the Prevention of Protein Adsorption by Steric Repulsion Theory, T. B. McPherson et al., Chapter 28 in PAI, op cit.: 395-404).

Short polymers covalently attached to a surface, which increase the interracial thickness and hydrophilicity and thus lower the availability of hydrophobic binding sites underneath, were shown to lower the probability for protein binding to, and denaturation at, the modified surface as well.

The fact that surfactants, which also often contain a short polymer segment at one end, tend to oppose or even partially reverse the binding of proteins to various surfaces is consistent with the above mentioned finding. The phenomenon probably involves protein solubilisation or replacement, depending on the relative strength of surfactant-surface interactions and surfactant-protein binding; usually both these factors play some role.

In another experiment, the addition of a Brij type non-ionic surfactant (an alkyl-polyoxyethylene ether) to the aqueous phase at pH 7.0 in the concentration range around 10⁻⁴ wt-% induced a substantial displacement of protein from the air/water interface (T. Arnebrant et al, op. cit.).

The removal of preadsorbed proteins by surfactants has been extensively studied (Protein-Surfactant Interaction at Solid Surfaces, T. Arnebrant et al. Chapter 17 in PAI, op. cit.: 240-254). Three types of interactions were discerned:

i) Binding of surfactant by electrostatic or hydrophobic interactions to specific sites in the protein, such as alpha-lactoglobulin or serum albumin;
ii) Co-operative adsorption of surfactant to the protein without gross conformational changes;
iii) Cooperative surfactant binding to the protein followed by conformational changes;

For example, removal of protein from methylated (hydrophobic) silica surfaces is similar for different surfactants, indicating that the proteins are removed through replacement due to higher surface activity of the surfactant. It may be concluded that surfactant headgroup effects are moat pronounced at hydrophilic surfaces but less important at hydrophobic ones (Protein-Surfactant Interaction at Solid Surfaces, T. Arnebrant et al., Chapter 17 in PAI, op. cit.,: 240-254).

Similar conclusions hold for the other lipids. The amount of plasma proteins adsorbed on a plastic surface decreases on pre-treatment with DPPC liposomes suspension; insulin adsorption on catheter surfaces reveals the same trend.

We have now unexpectedly found that amphipaths, especially macromolecules adsorb to soft surfaces comprising a mixture of lipids and surfactants more efficiently than to lipid aggregates containing no surface-active molecules. More generally speaking a blend of molecules forming a stable membrane—typically but not necessarily in the form of lipid vesicles (liposomes)- and at least one strongly amphipatic, that is, relatively water soluble, bilayer-destabilising component (often a surfactant), exemplified by a mixture of phospholipids and surfactants, is more prone to bind amphipaths, such as proteins than pure phospholipid surfaces, especially vesicles or liposomes which consist of phospholipids only or also comprise at least one bilayer stabilising lipid class substance, such as cholesterol. We have also found that the relative number of bound amphipathic macromolecules (proteins) is unexpectedly higher for the surfaces which bear net charges with the same sign as the net charge of the adsorbing entity. This is in clear contradiction within the published information, which teaches that electrostatic binding requires opposite charges on the interacting entities in order to be strong.

We propose that one of the requirements for the above stated improvement of supra-molecular (e.g. drug-carrier) association is the general adaptability of the adsorbent surface. This adsorption promoting capability permits the adsorbing macromolecules:
i) first, to get enriched near the adsorbent surface, due to the locally attractive charge-charge and other interactions;
ii) second, to optimise non-electrostatic interactions/binding to the adsorbent surface. (The latter process typically requires the presence of hydrophobic and H-bond binding sites, which are generated or made accessible by surface-flexibility and/or adaptability.)

(Macro-molecular) Drug-carrier combinations which fulfil these requirements—and permit their control—are best suited for practical applications.

We furthermore propose that each step involved in protein adsorption to a soft (membrane) surface depends, to a variable degree, on the proximity and numerosity of the hydrophobic binding sites in/at the membrane-solution interface. The kinetics of hydrophobic association between macromolecules and a binding surface, therefore, should be sensitive to the number of accessible binding sites which, in turn, is increased by the presence of surface-active ingredients in and softness of the membrane.

The rate at which adsorbing (macro)molecules can adjust conformationally to the multiple binding sites is important as well. For example, in the case of uncharged flexible (Transfersome®) membranes hydrophobic interaction is the main reason for insulin-surface association. The underlying multi-step binding usually requires substantial system rearrangements, however, and thus long adsorption time, to complete. Optimum incubation times for the formation of Transfersome®-insulin-complexes, consequently, may be rather long.

The adsorption scheme advocated in previous paragraphs agrees with the basic adsorption scenario described in specialised literature. This notwithstanding, several differences, and even controversies, clearly distinguish our findings from the public knowledge disclosed to date.

Unexpectedly, an addition of charged surfactants to a surface in accordance with the invention speeds up the process of protein binding to said surface and provides a means for controlling the extent and the rate of macromolecule-membrane association. This contradicts the above-mentioned, widely accepted, teachings that surfactants suppress protein binding. On the other hand, at least partial, surfactant elimination from such a surface accelerates the process of macromolecular desorption and sets some macromolecules free. This also directly opposes published knowledge.

Unexpectedly, we found that macromolecular adsorption to a soft deformable surface in accordance with the invention, especially a corresponding membrane, is stronger than to a less deformable surface. As pertinent literature claims that soft membranes are more hydrophilic and mutually repulsive man their less adaptable kind, this finding directly opposes expectation.

It is, therefore, one of the aims of our invention to specify the conditions which maximise the association between large, often macromolecular, amphipatic molecules, such as proteins, or any other kind of a suitable chain molecule, and a complex to adsorbent surface.

A further aim of the present invention is to define advantageous factors which control the rate of macromolecular adsorption to, or the corresponding rate of desorption from, a complex surface.

Yet another goal of our invention is to propose methods for preparing formulations suitable for (bio)technological and medicinal applications.

Another aim of this invention is to describe modalities which are particularly suitable for the practical use of resulting formulations; including, but not limited to, the use of resulting adsorbates in diagnostics, separation technology and (bio)processing, bioengineering, genetic manipulation, agent stabilisation, concentration or delivery, for example in medicine or veterinary medicine.

Solutions to these problems in accordance with present invention are defined in the attached independent claims.

Convenient solutions providing special advantages are defined in the subclaims.

### BRIEF DESCRIPTION OF THE DRAWINGS

FIG. 1 illustrates insulin adsorption on different ultra-deformable Transfersomes, as a function of protein/lipid concentration ratio in the bulk. In the lower panel, absolute bound protein amount is shown. In the upper panel, relative amount of vesicle associated insulin is shown. (Examples 1-27 A)

FIG. 2 presents the results of insulin binding experiments with ultra-deformable Transfersomes containing cholate as a function of total lipid concentration in the bulk. (Examples 1-27 B)

FIG. 3 presents the results of insulin binding experiments with ultra-deformable Transfersomes containing cholate, as a function of relative protein/lipid concentration in the bulk and of binding (incubation) time. (Examples 1-27 C)

FIG. 4 presents the results of insulin binding experiments with surfactant (cholate or Tween 80) containing Transfersomes, as a function of protein/lipid concentration ratio in the bulk. Absolute and relative amounts of bound protein are shown in the lower and upper panels, respectively. (Examples 46-59)

FIG. 5 presents the results of insulin binding experiments with Transfersomes, as a function of protein/lipid concentration ratio in the bulk. The insulin was provided from two different sources: (i) a solution made from dry, lyophilised human recombinant insulin power, and (ii) Actrapid™ commercially-available insulin solution (from Novo Nordisk). (Examples 72-76)

FIG. 6 presents the results of insulin binding experiments as a function of protein/lipid concentration ratio in the bulk with: (i) conventional liposomes (SPC), (ii) charged liposomes (SPC/SPG), and (iii) charged Transfersomes (SPC/SPG/Tween 80). (Examples 77-92)

FIG. 7 presents data on insulin association with extended surfaces of resulting vesicles as a function of increasing surface charge density, created by incorporating increasing relative amounts of charged phospholipid SPG into originally uncharged SPC/Tween (SPC/Tw) Transfersomes. (Examples 96-100)

FIG. 8 presents data on the effect of the method used to manufacture highly flexible charged membranes on insulin binding. (Examples 101-104)

FIG. 9 presents the results of insulin binding experiments using plain SPC liposomes as negative control and varying the following parameters: (i) ultra-deformable vesicle composition (SPC+ cholate; SPC+Tween 80); (ii) insulin kind/source (human recombinant insulin in Actrapid solution; lyophilized human insulin; porcine insulin in solution); and (iii) association (incubation) time (2 hours to 5 weeks).

FIG. 10 illustrates binding of interferon alpha on non-ionic (SPC/Tw80) and anionic (SPC/NaChol) ultradeformable vesicles as a function of protein/lipid concentration ratio in the bulk. (Examples 111-134)

FIG. 11 illustrates the change of blood glucose concentration over time after an epicutaneous administration of insulin associated with Transfersomes (Transfersulin). Dashed lines give 95% confidence limits.

FIG. 12 illustrates the change of blood glucose concentration over time in a normoglycaemic volunteer test person after administration of three different Transfersulin batches with identical Transfersomes, but different insulin batches. Open symbols give the result of negative control experiment.

### DEFINITIONS

An “associate”, by the definition used in this application, is a complex between two or more different molecules, at least one of which forms aggregates with one or several well defined surface(s), independent of the reason for complex formation but excluding covalent bonding. Association between different kinds of molecules can be based on encapsulation (e.g. enshrinement into a vesicle comprising the surface-forming molecule(s)), insertion (e.g. incorporation into the aggregate layer at and below the surface) or adsorption (onto the aggregate surface); combinations of two of more of these principles are also possible.

The terms “adsorbate”, “adsorbing (macro)molecule”, “binding (macro)molecule”, “associating (macro)molecule”, etc., in this application, are used interchangeably to describe an association between the molecules which do not form extended surfaces under the conditions chosen and an “adsorbent” or “binding surface”, etc., in the above mentioned sense.

“Carrier” means an aggregate, independent of the nature or source of its generation, which is capable to associate with one or more macromolecules used for practical purposes, such as an application on or the delivery into the human or animal body.

“Lipid”, in the sense of this invention, is any substance with characteristics similar to those of fats. As a rule, molecules of this type possess an extended apolar region (chain, X) and, in the majority of cases, also a water-soluble, polar, hydrophilic group, so called head-group (Y). Bask structural formula 1 for such substances reads

X—Yₙ (1)

where n is greater or equal zero. Lipids with n=0 are called apolar lipids; those with n≧1 are polar lipids. In the context of this text all amphiphiles, such as glycerides, glycerophospholipids, glycerophosphinolipids, glycerophosphonolipids, sulpholipids, sphingolipids, isoprenoidlipids, steroids, sterines or sterols, etc., and all lipids containing carbohydrate residues, are simply called lipids. For a more explicit definition we refer to PCT/EP 91/01596.

“Edge-active” substance or “surfactant”, in this application, refers to any substance which increases the system's propensity to form edges, protrusions or other strongly curved structures and defect-rich regions. In addition to common surfactants, co-surfactants and other molecules which promote lipid solubilisation in the presence of more conventional surfactants fall in this category; so do molecules which induce or promote the formation of (at least partly hydrophobic) defects in the adsorbent (hetero)aggregates. Direct surfactant action or indirect catalysis of (partial) molecular de-mixing, or else surfactant-induced conformation changes on relevant molecules are often responsible for the effect. Consequently, many solvents as well as asymmetric, and thus amphipatic, molecules and polymers, such as numerous oligo- and polycarbohydrates, oligo- and polypeptides, oligo- and polynucleotides and/or their derivatives belong in the above mentioned category in addition to conventional surfactants. A relatively extensive list of most popular standard surfactants, of some suitable solvents (otherwise called co-surfactants), and of many other relevant edge-active substances is found in PCT/EP 91/01596, to which we therefore refer here explicitly. A more complete list is found in Handbook of industrial surfactants; Michael Ash, Irene Ash, eds., Gower Publishing, 1993.

“Chain molecule” or “macromolecule” is any straight or branched chain molecule which contains at least two kind or states of group(s) with an unequal affinity for the “adsorbing surface”. The other requirement specific to the corresponding alternative (claim 2) or combined (claim 3) aspect of this invention is that at least one kind of such group must be (partially) charged in the donor solution and/or at the adsorbing surface. The surface-affinity difference for individual groups is often due to their different amphipaticity, that is, to the different hydrophilicity/hydrophobicity. Different groups can be distributed arbitrarily along the chain but, frequently, several physically related (e.g. several hydrophilic or more than one hydrophobic) groups are located in one chain segment.

“Macromolecules”, in the sense used in this application, include among others:

Carbohydrates, with a basic formula Cₓ(H₂O)_{y}, e.g. in sugar, starch, cellulose, etc. (for a more complete definition of carbohydrates we explicitly refer to PCT/EP 91/01596), for the purposes of this invention most often need to be derivatised to attain additional affinity for the binding surface. This can be done, for example, by attaching hydrophobic residues to the carbohydrates aimed to associate with a (partly) hydrophobic surface, or by introducing such groups that can participate in the other non-Coulombic (e.g. hydrogen bond) interactions with the more hydrophilic binding surface.

Oligo or polynucleotides, such as homo- or hetero-chains of deoxyribonucleic-(DNA) or ribonucleic acid (RNA), as well as their chemical, biological, or molecular biological (genetic) modifications (for a more detailed definition consider the lists given in PCT/EP 91/01596).

Oligopeptides or polypeptides comprise 3-250, often 4-100, and most often 10-50 equal or different amino acids, which are naturally coupled via amide-bonds, but in the case of proteomimetics may rely on different polymerisation schemes and may even be partly or completely cyclic; use of optically pure compounds or racemic mixtures is possible (see PCT/EP 91/01596 for a more explicit and complete definition).

Long polypeptidic chains are normally called proteins, independent of their detailed conformation or precise degree of polymerisation. Most, if not all, proteins associate rather efficiently with surfaces, as outlined in this work. We therefore refrain from quoting the relevant substances here and rather refer to PCT/EP 91/01596 for a partial list and to the specialised literature for the up-to-date listing.

For the purposes of illustration only, a few relevant classes are briefly summarised in the following.

Enzymes comprise oxidoreductases (including various dehydrogenases, (per)oxidases, (superoxid) dismutases, etc.), transferases (such as acyl-transferase, phosphorylase and other kinases), transpeptidases (such as: esterases, lipases, etc.), lyases (including—decarboxylases, isomerases, etc.), various proteases, coenzymes, etc.

Immunoglobulins from the classes of IgA, IgG, IgE, IgD, IgM with all subtypes, their fragments, such as Fab- or Fab2-fragments, single chain antibodies or parts thereof, such as variable or hypervariable regions, in the native form or chemically, biochemically or genetically manipulated can profit from this invention. This includes, but is not limited to, IgG-gamma chains, IgG-F(ab)2 fragments, IgG-F(ab), IgG-Fc fragments, Ig-kappa chains, light chains of Ig-s (e.g. a kappa and lambda chains) and also involves smaller immunoglobulins fragments, such as the variable or hypervariable regions, or modifications of any of these substances or fragments.

Immunologically active macromolecules other then antibodies (endotoxins, cytokines, lymphokines, and other large immunomodulators or biological messengers) also belong to the class of heterologous chain molecules. So do phytohaemagglutinins, lectins, polyinosine, polycytidylic acid (poli I:C), erythropoietin, “granulocyte-macrophage colony stimulating factor” (GM-CSF), interleukins 1 through to 18, interferons (alpha, beta or gamma and their (bio)synthetic modifications), tumour necrosis factors, (TNF-s); all sufficiently large and amphipatic tissue and plant extracts, their chemical, biochemical or biological derivatives or replacements, their parts, etc. All such molecules, consequently, can be associated conveniently and efficiently with complex surfaces as described in this document.

Further biologically relevant examples include substances that affect local or general growth, such as basic fibroblast growth factor (BFGF), endothelial cell growth factor (ECGF), epidermal growth factor (EOF), fibroblast growth factor (FGF), insulin, insulin-like growth factors (such as LGF I and LGF II), nerves-growth factors (such as NGF-beta, NGF 2,5s, NGF 7s, etc.), platelet-derived growth factor (PDGF), etc.

Derivatisations particularly useful for the purpose of this invention are the modifications, whether done (bio)chemically, biologically or genetically, by which adsorbates are substituted with several, often more than 3, apolar (hydrophobic) residues, such as an aryl, alkyl-, alkenyl-, alkenoyl-, hydroxyalkyl-, alkenylhydroxy- or hydroxyacyl-chain with 1-24 carbon atoms, as appropriate, or reactions through which the propensity for the formation of other non-Coulombic interactions between the adsorbate and the adsorbent increases. When macromolecules are hydrophobised, relatively small numbers (1-8, or even better, 1-4) of carbon atoms per side chain is advantageous. Pertinent scientific literature provides ample information on how chain molecules should be hydrophobised for different aims. For the purpose of this disclosure, strong anchoring of the adsorbent, which is covered by other publications (see e.g. Torchilin, V. P.; Goldmacher, V. S.; Smirnov, V. N. Biochem. Biophys. Res. Comm. 1978, 85: 983-990), is excluded not only due to its prior art nature but also since it is likely to result in poorly reversible association.

It is already known in the art that the addition of surfactants to a membrane built from an amphipatic substance modifies the adaptability of said membrane. Moreover, it has already been suggested that this fact may be used to improve agent transport through the otherwise confining pores in a barrier, by incorporating the agent into miniature droplets surrounded by the corresponding membranes and suspended in a suitable liquid medium. This is described in greater detail in our earlier applications PCT/EP 91/01596 and PCT/EP 96/04526.

The selections one has to make in order to optimise said vesicles with highly adaptable membranes for the purpose of barrier pores penetration are not generally identical with the steps one has to take to enable or to control the extent and the rate of association between a chain molecule, on the one hand, and such membranes, on the other hand. Furthermore, the three-dimensional adaptability of such membraneous surfaces, which surround said vesicles (and thus the deformability of the vesicle itself), is not necessarily relevant e.g. for associations process when said surface, with which a macromolecule is associating, is solid-supported, and therefore does not have the three-dimensional adaptability characteristic of non-supported membranes.

In order to enable and/or to control the processes of macromolecular association with a surface, on which this invention is focusing, two major effects can be employed, as already indicated above.

The first important phenomenon is that amphipatic molecules, namely the macromolecules or chain molecules already discussed, associate better with an extended surface which comprises at least one amphipatic substance, which tends to form extended surfaces, and at least one more substance, which is more soluble in the suspending liquid medium and also tends to form less extended surfaces man the former amphipatic substance. In other words, the presence of a substance with surface destabilising tendency renders surface-solution interface relatively more attractive for the adsorbing macromolecules compared with the corresponding surfaces formed from the less soluble surface-forming substance only, in the absence of the former more soluble, surface destabilising second substance. A surface, in the context of this document, is deemed to be extended if it allows propagation and/or evolution of co-operative surface excitations in two dimensions. The surface of a vesicle, for example, fulfils this criterion by supporting surface undulations or fluctuations; depending on membrane flexibility, average vesicle diameters between 20 nm and several hundred nanometres are needed for this. (Mixed) Lipid micelles, which do not reach this dimension at least in one direction, do not fulfil the requirement; if so, their surface is not considered to be extended in the sense of this invention.

The second, more soluble and surface-destabilising substance is generally an edge-active substance or surfactant.

The second newly disclosed effect is that, contrary to expectation, electrically charged macromolecules or chain molecules associate easier and better with an equally charged surface (i.e. both are negative or both are positive), when the latter is complex and comprises at least two amphipatic substances, one of which is more soluble than the other and also tends to destabilise the surface formed by the less soluble substance. In other words, while it is generally true that like charges repel each other; charged macromolecules or chain molecules; can associate with an equally charged surface better when either the associating substance and the substrate surface are negative, or else when both participants in the association process bear a net positive charge, provided that the surface complexity allows for the necessary intra- and inter-molecular rearrangements. Based on the existing wisdom, one would have expected the association to be easier and stronger in the case of negatively charged macromolecules associating with a positively charged surface, that is, when assisted by electrostatic attraction, and vice versa.

The two effects described in previous paragraphs can be advantageously combined, as is specifically defined in independent claim 3.

The selection of amphipatic, surface-forming substances can be defined in terms of differential solubility of participating substances, which together form the membrane or the surface, to which a macromolecule or a chain molecule is going to bind and which most often takes the form of vesicles suspended in a liquid medium. Generally, the inventive effect is more pronounced, i.e. the surface attractiveness for the binding macromolecule is higher, when the solubility difference between the participating molecules is greater. The more soluble membrane ingredient should be at least 10-fold, but preferably, at least 100-fold more soluble than the less soluble surface building component. Thus, when an amphipatic surface-forming substance, such as a phospholipid, is combined with a second substance, e.g. a surfactant, in a suitable liquid medium, such as water, it is much more advantageous to use a surfactant which is more soluble in water than the phospholipid (in right quantity) as the second component. On the other hand, the selection to be made can also be defined in terms of resulting surface curvatures. Using the above mentioned example of a phospholipid (as the basic surface-forming substance) mixed with a surfactant (as the surface-destabilising, more soluble second ingredient) in water (used as the liquid medium) the resulting vesicles attain some characteristic surface curvature. The (average) curvature is, generally speaking, defined as the inverse average radius of the areas enclosed by the surfaced under consideration. Generally, the addition of a surfactant will increase the curvature of mixed lipids vesicle surface compared to the curvature of phospholipid vesicles containing no surfactant. If there is a saturation concentration of the surfactant, which does not catastrophically comprise the curved surface stability, the optimum surfactant concentration is typically chosen to be below 99% of such saturation concentration; more often, the choice is between 1 and 80 mol-%, even more preferably between 10 and 60 mol-% and most preferably between 20 and 50 mol-% of the saturation concentration.

If, on the other hand, the saturation concentration in the respective system is inaccessible, owing to the fact that after surfactant addition the surface disintegrates before the saturation is reached, the amount of surfactant to be used is typically less than 99% of solubilising concentration. Again, the concentration optimum for the surfactant in the system is often between 1% and 80%, more often between 10 and 60% and preferably between 20 and 50% of the concentration limiting the formation of adsorbent surface, i.e. above the concentration at which the extended surface is replaced by a much smaller average surface, of the solubilised mixed lipid aggregates.

A convenient, practically useful blend of substances can be defined in terms of average curvatures of said surfaces as well. As is addressed in claim 7 the surfaces have an average curvature (defined as the inverse average radius of the areas enclosed by the surfaces) corresponding to an average radius between 15 nm and 5000 nm, often between 30 nm and 1000 nm, more often between 40 nm and 300 nm and most preferably between 50 nm and 150 nm. It should be stressed, however, that the curvature of adsorbent surface is not necessarily governed by the adsorbent membrane properties. When solid supported surfaces are used, and built according to this invention from a selected blend of amphipatic substances, the mean curvature of said surfaces is normally determined by the supporting solid surface curvature.

Furthermore, it is possible to express the invention in terms of relative concentration of the surface-related charged components, at least when the association between like charges is used. The relative concentration of such surface-related charged components is between 5 and 100 mol-%, more preferably between 10 and 80 mol-% and most preferably between 20 and 60 mol-%, of the concentration of all surface-forming amphipatic substances taken together. Expressed in terms of the net surface charge density, the surface is characterised by values between 0.05 Cb m⁻² (Coulomb per square metre) and 0.5 Cb m⁻², even better between 0.075 Cb m⁻² and 0.4 Cb m⁻², and best between 0.10 Cb m⁻² and 0.35 Cb m⁻².

It is preferable to select the concentration and composition of background electrolyte, which preferably comprises oligovalent ions, so as to maximise the positive effect of charge-charge interactions on the desired association. Generally, one keeps the bulk ionic strength between I=0.001 and I=1, preferably between I=0.02 and I=0.5 and even more preferably between I=0.1 and I=0.3.

Another useful definition of the invention focuses on adsorbent surfaces in the form of a membrane surrounding a tiny droplet of fluid. Such membranes are then often bilayer-like and comprise at least two kind or forms of (self-)aggregating amphiphilic substances with at least 10-fold, preferably at least 100-fold difference in the insolubility in a (preferably aqueous) liquid medium used to suspend the droplets. In such cases, the selection of substances which form the membrane can be specified by requesting that the average diameter of homo-aggregates of the more soluble substance or the diameter of hetero-aggregates comprising both substances is smaller than the average diameter of homo-aggregates containing merely the less soluble substance.

Total content of all amphipatic substances in the system, which are capable of forming a surface, is preferably between 0.01 and 30 weight-%, particularly between 0.1 and 15 weight-% and most preferably between 1 and 10 weight-% of total dry mass, especially where said combination is used to produce formulation to be applied on or in the human or animal body, for medical purposes mainly.

The surface-building or surface-supporting substance, i.e. the substance that is capable of forming extended surfaces, may advantageously be chosen amongst the biocompatible polar or non-polar lipids, especially when the adsorbent surface is to have a bilayer-like structure. Specifically, the main surface-forming substance may be chosen to be a lipid or a lipoid from any suitable biological source or a corresponding synthetic lipid, or else a modification of such lipids, preferably a glyceride, glycerophospholipid, isoprenoidlipid, sphingolipid, steroid, sterine or sterol, a sulphur- or carbohydrate-containing lipid, or any other lipid capable of forming bilayers, in particular a half-protonated fluid fatty acid, and preferably from the class of phosphatidylcholines, phosphatidylethanolamines, phosphatidylglycerols, phosphatidylinositola, phosphatidic acids, phosphatidylserines, sphingomyelins or sphingo-phospholipids, glycosphingolipids (e.g. cerebrosides, ceramidepolyhexosides, sulphatides, sphingoplasmalogens), gangliosides or other glycolipids or synthetic lipids, in particular of the dioleoyl-, dilinoleyl-, diunolenyl-, dilinolenoyl-, diarachidoyl-, dilauroyl-, dimyristoyl-, dipalmitoyl-, distearoyl, or the corresponding sphingosine-derivative type, glycolipids or diacyl-, dialkenoyl- or dialkyl-lipids.

The other, surface-destabilising and more soluble substance is advantageously a surfactant, and may advantageously belong to the class of nonionic, zwitterionic, anionic or canonic detergents; it is especially convenient to use a long-chain fatty acid or alcohol, an alkyl-tri/di/methyl-ammonium salt, an alkylsulphate salt, a monovalent salt of cholate, deoxycholate, glycocholate, glycodeoxycholate, taurodeoxycholate, or taurocholate, an acyl- or alkanoyl-dimethyl-aminoxide, esp. a dodecyl-dimethyl-aminoxide, an alkyl- or alkanoyl-N-methylglucamide, N-alkyl-N,N-dimethylglycine, 3-(acyldimethylammonio)alkanesulphonate, N-acyl-sulphobetaine, a polyethylen-glycol-octylphenyl ether, esp. a nonaethylen-glycol-octylphenyl ether, a polyethylene-acyl ether, esp. a nonaethylen-dodecyl other, a polyethyleneglycol-isoacyl ether, esp. a octaethyleneglycol-isotridecyl ether, polyethylene-acyl ether, esp. octaethylenedodecyl ether, polyethyleneglycol-sorbitane-acyl ester, such as polyethylenglykol-20-monolaurate (Tween 20) or polyethylenglykol-20-sorbitan-monocoleate (Tween80), a polyhydroxyethylene-acyl ether, esp. polyhydroxyethylene-lauryl, -myristoyl, cetylstearyl, or -oleoyl ether, as in polyhydroxyethylen-4 or 6 or 8 or 10 or 12, etc.-lauryl ether (as in Brij series), or in the corresponding ester, e.g. of polyhydroxyethylen-8-stearate (Myrj 45), -laurate or -oleate type, or polyethoxylated castor oil 40 (Cremophor EL), a sorbitane-monoalkylate (e.g. in Arlacel or Span), esp. sorbitane-monolaurate (Arlacel 20, Span 20), an acyl- or alkanoyl-N-methylglucamide, esp. in or decanoyl- or dodecanoyl-N-methylglucamide, an alkyl-sulphate (salt), e.g. in lauryl- or oleoyl-sulphate, sodium deoxycholate, sodium glycodeoxycholate, sodium oleate, sodium taurate, a fatty acid salt, such as sodium elaidate, sodium linoleate, sodium laurate, a lysophospholipid, such as n-octadecylene(oleoyl)-glycerophosphatidic acid, -phosphorylglycerol, or -phosphorylserine, n-acyl-, e.g. lauryl or oleoyl-glycero-phosphatidic acid, -phosphorylglycerol, or -phosphorylserine, n-tetradecyl-glycero-phosphatidic acid, -phosphorylglycerol, or -phosphorylserine, a corresponding palmitoeloyl-, elaidoyl-, vaccenyl-lysophospholipid or a corresponding short-chain phospholipid, or else a surface-active polypeptide.

The concentration of charged membrane components will often be in the relative range of 1-80 mol-%, preferably 10-60 mol-% and most preferably between 30-50 mol-%, based on the amount of all membrane-building components.

It is preferred that a phosphatidylcholine and/or a phosphatidylglycerol is chosen as the surface-supporting substance and a lysophospholipid, such as lysophosphatidic acid or methylphosphatidic acid, lysophosphatidylglycerol or lysophosphatidylcholine, or a partially N-methylated lysophosphatidylethanolamine, a monovalent salt of cholate, deoxycholate-, glycocholate, glycodeoxycholate- or any other sufficiently polar sterol derivative, a laurate, myristate, palmitate, oleate, palmitoleate, elaidate or some other fatty acid salt and/or a Tween-, a Myrj-, or a Brij-type, or else a Triton, a fatty-sulphonate or -sulphobetaine, —N-glucamide or -sorbitane (Arlacelo or Span) surfactant is chosen as the substance less capable of forming the extended surface.

It is advantagous that the average radius of the areas enclosed by said extended surfaces is between 15 nm and 5000 nm, often between 30 nm and 1000 nm, more often between 40 nm and 300 nm and most preferably between 50 nm and 150 nm.

Generally, the third kind of substance, which associates with the extended surface formed by the combination of the other two substances (and in case, a third, fourth, fifth, etc. substance, as required), can comprise any molecule with repeating subunits, especially in the form of chain molecules. Thus, the third substance can be an oligomer or a polymer. Especially, it can be an amphipathic macromolecular substance with an average molecular weight above 800 Daltons, preferably above 1000 Daltons and more often still above 1500 Daltons. Typically, such substances are of biological origin, or similar to a biological substance, and advantageously have biological activity, that is, are bio-agents.

The third (kind of) substance preferably associates with the invented membrane-like extended surfaces especially by becoming inserted into the interface (or interfaces) between the membrane and the liquid medium, such interface(s) being an integral part of said membranes.

The content of said third substance (molecules) or of corresponding chain molecules is generally between 0.001 and 50 weight-%, based on the mass of absorbent surface. Often, the content is between 0.1 and 35 weight-%, more preferably between 0.5 and 25 weight-% and mostly between 1 and 20 weight-%, using similar relative units, whereby the specific ratio often is found to decrease with increasing molar mass of said adsorbing (chain) molecules.

Whenever the adsorbing macromolecule or chain molecule is a protein, or a part of protein, it is generally found that such entity can associate in the sense of this invention with the adsorbing surface, provided that it comprises at least three segments or functional groups with a propensity to bind to the adsorbent surface.

The macromolecules or chain molecules which, in accordance with the present invention, tend to associate with an extended surface formed from said amphipats may belong to the class of polynucleotides, such as DNA or RNA, or of polysaccharides, with at least partial propensity to interact with the surface, be it in their natural form or after some suitable chemical, biochemical or genetic modification.

The chain molecules associating with an extended surface may have a variety of physiological functions and act, for example, as an adrenocorticostaticum, a β-adrenolyticum, an androgen or antiandrogen, antiparasiticum, anabolicum, anaestheticum or analgesicum, analepticum, antiallergicum, antiarrhythmicum, antiarteroscleroticum, antiasthmaticum and/or bronchospasmolyticum, antibioticum, antidrepressivum and/or antipsychoticum, antidiabeticum, an antidote, antiemeticum, antiepilepticum, antifibrinolyticum, anticonvulsivum, an anticholinergicum, an enzyme, coenzyme or a corresponding inhibitor, an antihistaminicum, antihypertonicum, a biological inhibitor of drug activity, an antihypotonicum, anticoagulant, antimycoticum, antimyasthenicum, an agent against Morbus Parkinson or Morbus Alzheimer, an antiphlogisticum, antipyreticum, antirheumaticum, antisepticum, a respiratory analepticum or a respiratory stimulant, a broncholyticum, cardiotonic, chemotherapeuticum, a coronary dilatator, a cytostaticum, a diureticum, a ganglium-blocker, a glucocorticoid, an anti-flue agent, a haemostaticum, hypnoticum, an immunoglobuline or its fragment or any other immunologically active substance, a bioactive carbohydrate(derivative), a contraceptive, an anti-migraine agent, a mineralocorticoid, a morphine-antagonist, a muscle relaxant, a narcoticum, a neurotherapeuticum, a neurolepticum, a neurotransmitter or some of its antagonists, a peptide(derivative), an ophthalmicum, (para)-sympaticomimeticum or (para)sympathicolyticum, a protein(derivative), a psoriasis/neurodermitis drug, a mydriaticum, a psychostimulant, rhinologicum, any sleep-inducing agent or its antagonist, a sedating agent, a spasmolyticum, tuberculostaticum, urologicum, a vasoconstrictor or vasodilatator, a virustaticum or any of the wound-healing substances, or any combination of such agents.

The invention also can be used advantageously when the third substance is a growth modulating agent.

Further examples of advantageous embodiments include third substances selected from the class of immuno-modulators, including antibodies, cytokines, lymphokines, chemokines and correspondingly active parts of plants, bacteria, viruses, pathogens, or else immunogens, or parts or modifications of any of these, enzymes or co-enzymes or some other kind of a bio-catalyst; a recognition molecule, including inter alia adberins, antibodies, catenins, selectins, chaperones, or parts thereof, a hormone, and especially, insulin.

In the case of insulin, the invented combination preferably contains 1 through 500 I.U. of insulin per millilitre, in particular between 20 and 400 I.U. of insulin per millilitre and most preferably between 50 and 250 I.U. of insulin per millilitre, as the active substance. The preferred form of drug is human recombinant insulin or humanised insulin.

Other advantageous uses of the present invention include the application of various cytokines, such as interleukines or interferons etc., said interleukines being suitable for the use in humans or animals, including IL-2, IL-4, IL-8, IL-10, IL-12, said interferons being suitable for the use in humans or animals, including but not restricted to IF alpha, beta and gamma.

Said combination contains between 0.01 mg and 20 mg interleukin/mL, in particular between 0.1 and 15 mg and most preferred between 1 and 10 mg interleukin/mL, if necessary after a final dilation to reach the practically desirable drug concentration range.

Said combination contains up to 20 relative wt-% interferon, in particular between 0.1 and 15 mg interferon/mL and most preferred between 1 and 10 mg interferon/mL, if necessary after a final dilution that brings the drug concentration into practically preferred concentration range.

In another embodiment of present invention, the administration of nerve growth factor (NGF), associated as the (third), active substance with the invented surfaces, is described. The preferred form of such an agent is human recombinant NGF, optimum concentration ranges for the application contain up to 25 mg nerve growth factor (NGF) /mL suspension or up to 25 relative w-% of NGF as an agent, especially 0.1-15 rel. w-% protein and most preferred between 1 and 10 rel. wt-% NGF and, if needed, diluted before use.

It is possible to use the invented technology herein repeated for the purpose of immunoglobulin (Ig) administration, in the form of intact antibodies, parts of antibodies or some other biologically acceptable and active modification thereof. It is advantageous if the suspension contains up to 25 mg of immunoglobulin(Ig)/mL suspension or up to 25 w-% of Ig relative to total lipid, preferably with 0.1 rel. w-% to 15 rel. w-% protein and most advisable with 1 rel. w-% to 10 rel w-% immunoglobulin.

The invention discloses methods of preparing the above-defined combinations, especially as formulations of an active agent, especially a biologically, cosmetically and/or pharmaceutically active agent as discussed above, such methods comprising the selection of at least two amphipatic substances which differ in their solubility in a suitable liquid medium and which, at least when combined, are capable of forming an extended surface, especially in the form of a membrane, in the contact with said medium. It is a recommended selection criterion for these methods to use an extended surface formed by combining substances capable of attracting the active agent and supporting the association with said surface, provided that said surface is more attractive for the agent than the surface formed from merely that of the two substances which forms more extended surfaces on its own than the other substance on its own, and/or selecting at least two amphipatic substances, which differ in their solubility in a suitable liquid medium, provided that such substances, at least when combined, are capable of forming an extended surface, especially a membrane-like surface, in contact with said medium, and further provided that said surface comprising a combination of both substances is more attractive for and is better capable of binding active agent than the surface formed from that of the two substances alone which forms more extended surfaces than the other substance, and last but not least provided, in case that the surface as well as the agent bear a net electric charge, that the surface as well as the agent are both negatively charged or else are both positively charged, on the average.

Preferred methods for preparing invented extended surfaces include mechanical operations on a corresponding mixture of substances, such as filtration, pressure change or mechanical homogenisation, shaking, stirring, mixing, or by means of any other controlled mechanical fragmentation in the presence of the agent molecules which are to associate with the surface formed in the process.

It is preferred if the selected combination of surface forming substances is permitted to adsorb to, or in some other way is brought into permanent contact with, (a) suitable supporting solid surface(s), and then with the liquid medium by adding one substance after another or several at a time, whereby at least one of the later surface-forming steps is carried out in the presence of the agent that subsequently associates with the solid-supported surface.

It is advantageous if the adsorbing surfaces or their precursors, whether suspended in a liquid medium or supported by a solid, are first prepared by steps which may include sequential mixing of the surface forming molecules, and the associating molecules are then added and permitted to associate with the said surfaces, if necessary assisted by agitation, mixing or incubation, provided that such treatment does not break-up the preformed surfaces.

It is a preferred method of this invention to prepare formulations for non-invasive application of various agents, especially through the intact skin of humans or animals or plants, to create surfaces capable of associating with the agent molecules in complexes comprising at least one amphiphilic substance, at least one hydrophilic fluid, at least one edge-active or surfactant substance, and at least one agent. Together, these ingredients give rise to a formulation suitable for non-invasive agent application whereby other customary ingredients may also be added as suitable and necessary for achieving the desired properties and stability of the final preparation.

In operating the method, one may advantageously mix the selected ingredients separately and, if required, co/dissolve the components in a solution, then combine the resulting mixture(s) or solution(s) and finally to induce the formation of agent-binding entities or surfaces, preferably by the action of mechanical energy, as already explained.

Amphiphilic substances suitable for the purpose as disclosed in the present invention may be used either as such, or dissolved in a physiologically compatible polar fluid, such as water, or miscible with such solvent, or in a solvation-mediating agent together with the polar solution which then preferably comprises at least one edge-active substance or a surfactant.

One preferred way of inducing the formation of agent-attracting surfaces is by substance addition into the fluid phase. Alternatives include evaporation from a reverse phase, injection or dialysis, or exerting mechanical stress, e.g. by shaking, stirring, vibrating, homogenisation, ultrasonication (i.e. an exposure to ultrasonic waves), shear, freezing and thawing, or filtration under convenient and suitable driving pressure. When filtration is used, the filtering material may advantageously be chosen to have pore sizes between 0.01 μm and 0.8 μm, preferably between 0.02 μm and 0.3 μm, and most preferably between 0.05 μm and 0.15 μm. Several filters may be used sequentially or in parallel, as appropriate, in order to achieve the desired surface formation effect and to maximise the ease and speed of manufacturing.

It is advantageous if said agents and carriers are made to associate, at least partly, after formation of the adsorbing surface.

It is possible to form associates between the agent molecules and binding surfaces immediately before applying the resulting formulation for practical purposes. One may then start with a suitable concentrate or a lyophilisate.

The invention discloses preparation of agent carriers, especially for the purpose of drug delivery, drug depots, or any other kind of medicinal or biological application. Thus, it is possible to use the invention also in the context of barrier pore penetration; in this case, one will advantageously provide the associating surface in the form of a membrane formed by amphipatic molecules surrounding miniature droplets, as already known in the art, with the agent molecules associating with said droplet surface, to be carried by said ultra-deformable droplets through the pores in a barrier, even when the average diameter of the barrier pores is less, even much less, than the average diameter of droplets or vesicles. It may be necessary, however, to compromise between optimum association properties, on the one hand, and the best membrane adaptability properties, on the other hand, since the two, as was already explained above, are not necessarily the same, and more often than not actually differ from optimum composition properties defined by the vesicle membrane adaptability to the pore passage alone.

Further uses of the invented associates comprise bio-engineering applications, genetic manipulations, but also applications in separation technology, for (bio)processing or for diagnostic purposes. Here, as in the other invented uses, including enzymatic processes and catalysis, it can be useful to employ the aspect of the invention according to which the associating surface may be solid supported, rather than taking the form of a membrane vesicle. This allows the invented surfaces to be fixed to a solid support, which is then conveniently treated, attached, separated, concentrated, etc., for example with the intent to fix catalytically active macromolecules associated with this kind of surface to the maximum possible extent on the solid support. It is possible to stabilise surface-associating molecules, especially chain molecules, that are at least partially amphipatic, such as (derivatised) proteins, polypeptides, polynucleotides, or polysaccharides and/or in catalysing processes which involve such molecules in the surface-associated state. It is, therefore, conceivable to use the teaching of the present invention in order to prepare, say, columns packed with catalytically active, highly affine or selective, or otherwise reactive macromolecules. One example for this are chemical reactions done by passing suitable co-reactant(s), e.g. in a solution, through the column comprising solid-supported surfaces with the active molecules non-covalently attached, and thus surround the solid support, where the reaction with said active macromolecules takes place, as the solution passes the immobilised macromolecules. In another illustrative example, a solution of molecules at least some of which should be segregated from the solution is passed through a column filled with or is brought into contact with the suspension of solid-supported adsorbent surfaces with the aim of first letting the target molecules to associate with the substrate surface and then separating the fluid and solid compartments by any suitable method, including but not limited to centrifugation, sedimentation, floating (both with or without centrifugation) electrical or magnetic adsorbent particle segregation, etc.

Another use of the present invention relates to the control of kinetics and/or the reversibility of association or dissociation between said surface-associating molecules, on the one hand, and the complex, adaptable surface, as formed in accordance with this invention, by combining suitable amphipatic substances, whereby the higher surface charge density and/or the greater surface softness and/or the higher surface defect density can be used to speed up the association. A corresponding reduction may then be used to slow down the rate of association, or else to induce partial or complete dissertation.

Formulation and storage temperature seldom falls outside the range 0° C. to 95° C. Owing to the temperature sensitivity of many interesting ingredients, especially of many macromolecules, temperatures below 70° C. and even better below 45° C. are preferred. The use of non-aqueous solvents, cryo- or heat-stabilisers may allow working in different temperature ranges. Practical application is typically done at room or at physiological temperature, but usage at different temperatures is possible and may be desirable for specific formulations or applications. Maintenance of the adsorbing surface adaptability (flexibility, charge sign and/or charge density) at higher temperatures is one possible reason for this; keeping the agents in an active form at low temperatures provides another possible example.

Formulation characteristics are reasonably adapted to the most sensitive system component. Storage in the cold (e.g. at 4° C.) may be advantageous as well as the use of an inert atmosphere (e.g. nitrogen).

The disclosed formulations can be processed at the site of application using procedures specific for the adsorbent or adsorbate, whichever is more important. (Examples of adsorbents based on phospholipids are found in: “Liposomes” (Gregoriadis, G., ed, CRC Press, Boca Raton, Fla., Vols 1-3, 1987); ‘Liposomes as drug carriers’Gregoriadis, G., ed., John Wiley & Sons, New York, 1988; Liposomes. A Practical Approach, New, R., Oxford-Press, 1989). The formulation also can be diluted or concentrated (e.g. by ultracentrifugation or ultrafiltration).

In due time or before formulation use, the additives can be introduced to improve the chemical or biological stability of resulting formulation, the (macro)molecular association or its reversal, the kinetics of de/association, the ease of administration, compliance, etc.

Interesting additives include various system optimising solvents (the concentration of which should not exceed the limits defined by maintaining or reaching desirable system characteristics, chemical stabilisers (e.g. antioxidants, and other scavengers), buffers, etc., adsorption promotors, biologically active adjuvant molecules (e.g. microbicides, virustatics), etc.

Solvents suitable for the above mentioned purpose include, but are not limited to, the unsubstituted or substituted, e.g. halogenated, aliphatic, cycloaliphatic, aromatic or aromatic-aliphatic carbohydrates, such as benzole, toluol, methylenechloride, dichloromethane or chloroform, alcohols, such as methanol or ethanol, propanol, ethyleneglycol, propanediol, glycerol, erithritol, short-chain alkanecarbon acidesters, such as acetic adic, acidalkylesters, such as diethylether, dioxane or tetrahydrofurane, etc. and mixtures thereof.

It may also be convenient to adjust the pH-value of adsorbent/adsorbate mixture after its preparation or just prior to its use. This should prevent deterioration of individual system components and/or associates. It also should improve the biological activity or physiological compatibility of resulting mixture. To neutralise the mixture for the biological applications in vivo or in vitro, biocompatible acids or bases are often used to bring pH-value between 3-12, frequently 5 to 9 and most in the range between 6 and 8, depending on the goal and site of application. Physiologically acceptable acids are, for example, diluted aqueous solutions of mineral acids, such as hydrochloric acid, sulphuric acid, or phosphoric acid, and organic acids, such as carboxyalkane acids, e.g. acetic acid. Physiologically acceptable bases are, for example, diluted sodium hydroxide, suitably ionised phosphoric acids, etc.

All implicitly and explicitly mentioned lipids and surfactants are known. Lipids and phospholipids which form aggregates suitable of association with macromolecules are surveyed, for example, in ‘Phospholipids Handbook’ (Cevc, G., ed., Marcel Dekker, New York, 1993). ‘An Introduction to the Chemistry and Biochemistry of Fatty acids and Their Glycerides’ (Gunstone, F. D., ed.) and in other reference books. A survey of commercial surfactants is given in the annals ‘Mc Cutcheon's, Emulsifiers & Detergents’, (Manufacturing Confectioner Publishing Co.) and in other pertinent reference books (such as Handbook of Industrial Surfactants, M. Ash & I. Ash, eds., Gower, 1993). Relevant compilations of actives are, for example, ‘Deutsches Arzneibuch’, The British Pharmaceutical Guide, European Pharmacopoeia, Japanese Pharmacopoeia, The United States Pharmacopoeia, etc. Relevant macromolecules are described in manufacturers catalogues, pertinent scientific periodicals and specialised reference books, both from industry and academia.

This application describes some relevant properties of associates, as exemplified with a few selected polypeptide/protein and phospholipid/surfactant mixtures. The validity of general conclusions is not restricted to the presented choices, however, nor are the resulting associates solely useful in the field of human and veterinary medicine.

The following examples should illustrate the invention without setting or delineating its limits. All temperatures are in degree Celsius, carrier sizes are in nanometres, ratios and percentages are given in molar units. Otherwise, standard SI units are used, unless differently suited.

### EXAMPLES

The following experiments were performed to determine the binding capacity of insulin on complex vesicles. Different compositions vesicle compositions were used. The variations included different surfactant and lipids to introduce net charges onto/into the vesicles, different lipid/detergent ratios, different total lipid contents and various insulin kinds and concentrations.

In the first series of experiments, complex lipid vesicles comprising a phospholipid/biosurfactant mixture were combined with insulin at different protein/lipid ratios to find the binding maximum. Conventional, single component vesicles (liposomes) were used for reference.

### Examples 1-27

Ultradeformable and flexible vesicles (Transfersomes™):
Starting suspension
Total lipid (TL) content 10 w-% comprising:
   874.4 mg phosphatidylcholine from soy-bean
   125.6 mg sodium cholate
   9 mL phosphate buffer, pH 7.1
Final suspension A
TL content 5 w-%, comprising
   lipids as above and
   0.1, 0.5, 1, 2, 3, 4 mg insulin per 100 mg TL

To achieve the desired dilutions, the stock solution of insulin (4 mg/mL Actrapid™ Novo-Nordisk) was mixed with the buffer as follows:

| for: | | | insulin solution | |
|---|---|---|---|---|
| mg Insulin/100 | | | (4 mg/mL; | |
| mg Lipid | Buffer | | Actrapid) | |
| 4 | — | | 3 | mL |
| 3 | 0.75 | mL | 2.25 | mL |
| 2 | 1.5 | mL | 1.5 | mL |
| 1 | 2.25 | mL | 0.75 | mL |
| 0.5 | 2.265 | mL | 0.375 | mL |
| 0.1 | 2.925 | mL | 0.075 | mL |

Final suspensions A were prepared by mixing 2.5 mL of the starting lipid suspension (10% TL) and 2.5 mL of the appropriate insulin dilution.
Final suspension B
TL content 5 w-% to 0.25 w-%, comprising
   lipids as given above and
   4, 5, 6.67, 10, 20, 40, and 80 mg insulin per 100 mg TL

To get the different insulin/lipid ratios, the following pipetting scheme was used:

| | | | |
|---|---|---|---|
| for: | achieved | starting | |
| mg insulin/100 | final TL | suspension | |
| mg lipid | (w-%) | (10% lipid) | buffer |

| 4 | 5 | 3 | mL | — | |
|---|---|---|---|---|---|
| 5 | 4 | 2.4 | mL | 0.6 | mL |
| 6.67 | 3 | 1.8 | mL | 1.2 | mL |
| 10 | 2 | 1.2 | mL | 1.8 | mL |
| 20 | 1 | 0.6 | mL | 2.4 | mL |
| 40 | 0.5 | 0.3 | mL | 2.7 | mL |
| 80 | 0.25 | 0.15 | mL | 2.85 | mL |

Final suspensions B were prepared by mixing 2.5 mL Actrapid HM (4 mg/mL insulin) with 2.5 mL of an appropriately diluted lipid suspension.
Final suspension C
TL content 2.5 w-% to 0.125 w-%, comprising
   lipids as given above and
   4, 5, 6, 7, 8, 9, 10, 15, 20, 30, 40, 50, 80 and 160 mg insulin per 100 mg
   TL

To get the quoted insulin/lipid ratios, the following pipetting scheme was used:

| | | starting lipid | | | | | |
|---|---|---|---|---|---|---|---|
| for: | final TL | suspension, | | insulin solution | | | |
| insulin/100 | conc. | diluted to | | (4 mg/mL; | | | |
| mg lipid | (w-%) | 5 w-% lipid | | Actrapid) | | buffer | |
| 4 | 2.5 | 2.5 | mL | 1.25 | mL | 1.25 | mL |
| 5 | 2.5 | 2.5 | mL | 1.563 | mL | 0.938 | mL |
| 6 | 2.5 | 2.5 | mL | 1.875 | mL | 0.625 | mL |
| 7 | 2.5 | 2.5 | mL | 2.188 | mL | 0.313 | mL |
| 8 | 2.5 | 2.5 | mL | 2.5 | mL | — | |
| 9 | 2.2 | 2.222 | mL | 2.5 | mL | 0.278 | mL |
| 10 | 2 | 2 | mL | 2.5 | mL | 0.5 | mL |
| 15 | 1.3 | 1.333 | mL | 2.5 | mL | 1.167 | mL |
| 20 | 1 | 1 | mL | 2.5 | mL | 1.5 | mL |
| 30 | 0.67 | 0.667 | mL | 2.5 | mL | 1.833 | mL |
| 40 | 0.5 | 0.5 | mL | 2.5 | mL | 2 | mL |
| 50 | 0.4 | 0.4 | mL | 2.5 | mL | 2.1 | mL |
| 80 | 0.25 | 0.25 | mL | 2.5 | mL | 2.25 | mL |
| 160 | 0.125 | 0.125 | mL | 2.5 | mL | 2.375 | mL |

For the test series C, a 5% vesicle suspension was prepared from the 10% stock suspension, by diluting the suspension 1:1 vol:vol with buffer and repeating the filtering and freeze-thawing procedure as described below.

Preparation of adsorbent/adsorbate mixture. Buffer was prepared by the standard procedures and filtered through a 0.2 micrometer sterile filter. (For future use, the solution was stored in a glass container.) Lipid mixture was suspended in the buffer in a sterile glass container, covered tightly, and stirred on a magnetic stirrer for 2 days at room temperature. The suspension then was extruded sequentially through the etched-track polycarbonate membranes (Nucleopore type) with the nominal pore size of 400 nm, 100 nm, and 50 nm, respectively. 3 passes were made each time, using driving pressures between 0.6 MPa and 0.8 MPa. The resulting vesicle suspension was frozen and thawed 5 times at the respective temperatures of −70° C. and +50° C. To get the desired final vesicle size, the suspension was re-extruded, 4 times through a 100 nm filter at 0.7 MPa. As a last step, the highly deformable vesicles were sterilised by a filtration through a sterile syringe filter with 200 nm pores. Vesicles were stored in sterile polyethylene containers at 4° C. prior to use.

Each insulin molecule carries a net negative charge in the neutral pH region, owing to the excess of negatively charged amino acids over the positively charged amino acids above pI=5.4.

Commercially available insulin solution (Actrapid™ from Novo-Nordisk) was used for many, including this, association study. Consequently, the starting protein solution contained 4 mg insulin/mL and 3 mg m-cresol/mL. By adding an appropriate amount of such solution to the suspension of adsorbent vesicles different nominal insulin/lipid ratios were generated. The resulting carrier-insulin mixtures were carefully but thoroughly mixed and incubated for at least 2 hours, depending on the experiment, at room temperature.

In the test series A, the final suspension was prepared by diluting the original vesicle suspension with Actrapid to obtain final lipid concentration of: 50 mg TL/mL and different protein/lipid ratios. In the test series B, the final lipid concentration varied between 2.5 mg/mL and 40 mg/mL, depending on the insulin/TL ratio. In the test series C, the final lipid concentration ranged from 1.25 to 25 mg/mL. For comparison, similar dilution series was prepared by using buffer instead of the lipid suspension.

Test measurements were done with 4 mL of insulin/vesicle mixture each. After 2 hours, lipid vesicles were separated from the aqueous sub-phase in order to determine how much insulin (in whichever way) has associated with the lipid vesicles, and how much remained unbound in the water sub-phase. For this purpose, CENTRISART I-ultracentrifugation tubes with a cut-off of 100.000 Da were used. Three tubes were used for each dilution with 1 mL of the insulin containing suspension and were centrifuged at 2000 g for 3 hours (T= 10° C.). Insulin concentration in the resulting, optically clear supernatant (assumed to contain merely buffer, insulin and some mixed lipid (phosphatidylcholine/cholate) micelles together with the dissolved detergent was determined. Supernatants mat were NOT optically clear were discarded as it has been shown that such supernatants were contaminated with lipid vesicles that had passed through the defects in CENTRISAT I filters. Standard HPLC procedure was used for all insulin determinations reported herein. Measurements were done in duplicate.

Original dilutions served as positive controls. In negative controls, the non-specific insulin adsorption to the test device was quantified. After correction for such non-specific binding, the difference between starting and final insulin concentration in the supernatant was calculated. The “missing” insulin was assumed to be associated with the vesicles and expressed in absolute or relative terms.

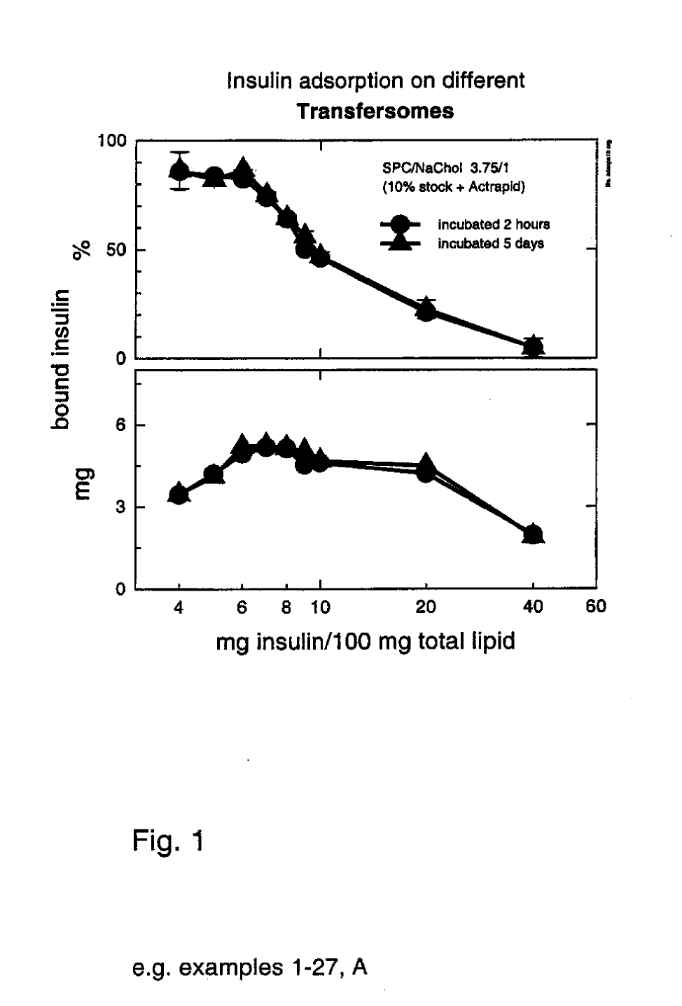

Results of the above described experiment are given in FIG. 1. They suggest that below insulin/lipid ratio of 6 mg/100 mg TL, 80-90% of protein added associates with (binds to) the vesicles. At higher insulin/lipid ratios, the relative efficiency of protein-surface association decreases, to reach only 5% binding for ⅖ (40 mg/100 mg) dilution. In other words, 2 mg of each 40 mg insulin added at a high dilution and at high protein/lipid ratio tends to associate with (nominally) 100 mg lipid in the form of highly deformable vesicles.

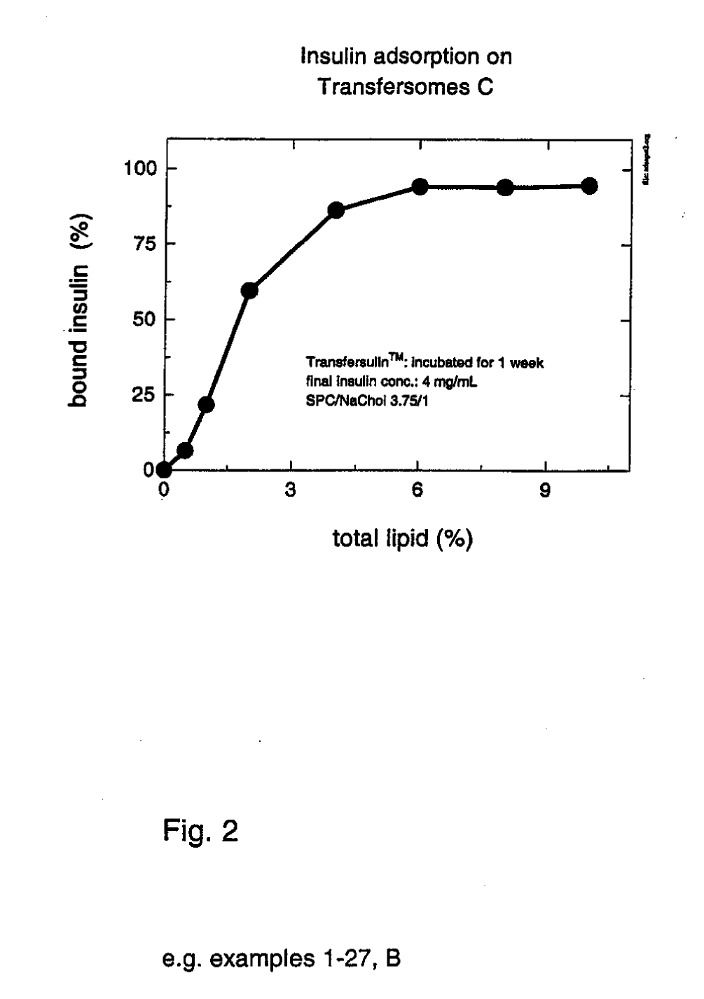

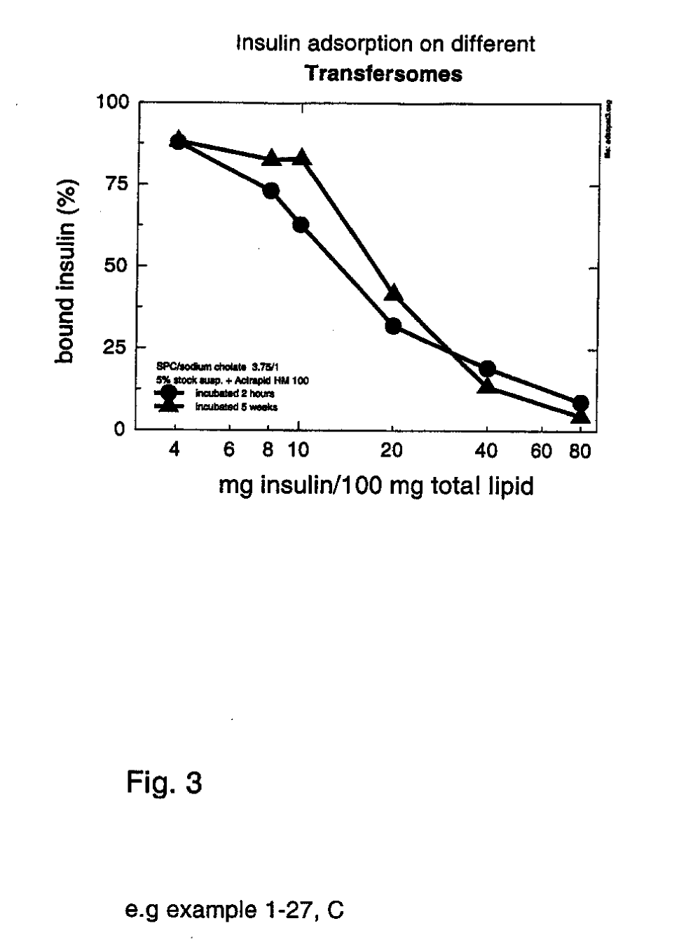

Prolonging incubation time or, to a lesser extent, increasing the added suspension concentration improves the situation (FIGS. 2 and 3).

### Examples 28-45

Standard vesicles (liposomes), starting suspension:
1 g phosphatidylcholine from soy-bean
9 mL phosphate buffer, pH 7.1

Final suspension A
TL content 5 w-%, comprising
lipids as above and
0.1, 0.5, 1, 2,3,4 mg insulin per 100 mg TL
(0.1, 0.5, 1, 2, 3, 4 rel. w-%)

Final Suspension B
TL content 5 w-% to 0.25 w-%, comprising
lipids as given above and
4, 5, 6.67, 10, 20, 40, and 80 mg insulin per 100 mg TL

Starting lipid suspension was made as described for examples 1-27. However, in order to obtain sufficiently monodisperse preparation of small enough liposomes, 6 additional extrusions through 100 nm filters had to be made.

Insulin binding to the tested liposomes was found to be very low. Only 2% to 5% of the added drug have combined with the standard lipid vesicles in the 4 mg/mL to 100 mg/mL-dilution range (data not shown graphically).

To check, and experimentally exclude, the effects of suspension dilution on the composition of highly deformable complex vesicles the following experiments were done.

### Examples 46-59

Starting suspension:
874.4 mg phosphatidylcholine from soy-bean
125.6 mg sodium cholate (giving 10 V-% TL content)
9 mL phosphate buffer, pH 7.1

Final suspension:

The composition of final suspensions was the same as in series B and C of examples 1-27, including decreasing final lipid concentrations.

Measured insulin/lipid ratios were: 4, 8, 10, 20, 40, 80,160 mg insulin per 100 mg TL

Preparation of the vesicle suspension complies with the description given for examples 1-27 for the stated insulin/lipid ratios, except in that the dilutions were made either with Actrapid containing 10 mM cholate and/or buffer containing 5 to 20 mM cholate (for the control and test samples). This was done so that the final cholate concentration in all samples was 5 mM, which is close to the CMC of this detergent, to prevent cholate dissociation from the vesicles membrane after dilution.

By preventing cholate washout from the vesicles, not only the original actual vesicle composition but also the average charge density of vesicle surface was maintained.

These improvements were reflected in the binding

In the examples of this test series, we took especial care to keep nominal cholate concentration below 5 mM stage throughout the pipetting process, to prevent an inadverted vesicle solubilisation, which is particularly likely in the range of low total lipid concentrations.

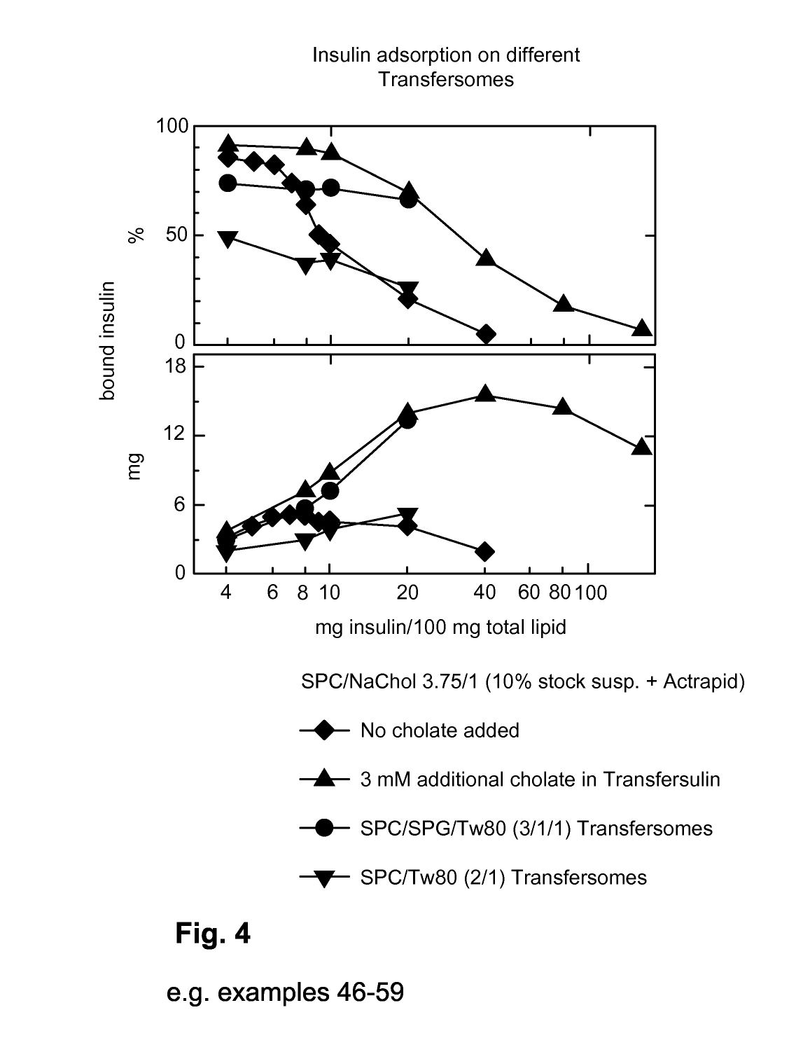

Results show that up to the protein/lipid weight ratio of 10%, between 80% and 90% of the added insulin bind to the lipid vesicle surface (FIG. 4). This means that adsorbent-adsorbate association is almost perfect and the efficiency of protein binding very high. The percentage of lipid associated protein decreases slowly with increasing protein/lipid ratio and reaches 7% at 1.6 mg insulin/1 mg lipid.

Absolute amount of the carrier-associated insulin reaches a maximum at approximately 0.4 mg insulin per 1 mg lipid, where 15.6 mg of the added 40 mg insulin are found to have associated with 100 mg total lipid in the form of highly deformable vesicles. Best yield is obtained at relative ratio 0.2 mg insulin per 1 mg total lipid, however, where 14 mg of the added 20 mg are measured to have associated with the mixed lipid vesicles. FIG. 4 illustrates these data.

Similar results are obtained if the cholate molecules are introduced into the mixed lipid vesicles suspension with the buffer or insulin solution.

### Examples 68-71

Starting suspension (20% TL):
1099.7 mg phosphatidylcholine from soy-bean
900.3 mg Tween 80
8 mL phosphate buffer, pH 7.4

Final suspension comprising:
lipid mixture as given above
2, 4, 8, 10, 20, and 40 mg insulin per 100 mg TL

Preparation of vesicle suspension was done essentially as described in examples 1-27 except in that stirring time was extended to 7 days. Actrapid™ (Novo-Nordisk) was the source of adsorbing insulin in all cases.

In order to be able to use fixed insulin concentration of 4 mg/mL, insulin/lipid ratios with the changing final total lipid concentration between 8 mg/mL and 100 mg/mL were prepared. For comparison (regarding a possible dilution effect), vesicles of similar composition were used to prepare different insulin/lipid ratios but with a fixed final total lipid concentration of 10 mg/mL (1 w-%). Protein-vesicle association time was chosen to be 3 hours.

The centrifugation time used to separate the non-associated insulin from the vesicle bound protein was an 6 hours (at 1000 g). Ail other experimental details were the same as in the first test series (examples 1-27).

Results. Aside from the fact that insulin binding to the membranes that contain nonionic surfactant (Tween-80) is generally lower than to the charged (cholate containing) membranes the qualitative characteristics of both adsorbent systems are similar (see examples 1-27.

Insulin association with the membranes at relative insulin/lipid ratio 0.04 mg insulin/1 mg lipid is approximately 50%. Relative concentration 0.2 mg insulin/1 mg lipid maximum binding corresponds to only 5.2 mg bound protein of the totally added 20 mg insulin. Absolute optimum, that is, the best yield in this test series, is obtained with 0.04 mg insulin/1 mg lipid.

### Example 72-76

Starting suspension (10% TL) comprising:
874.4 mg phosphatidylcholine from soy-bean
125.6 mg sodium cholate
9 mL phosphate buffer, pH 7.1 (−7.4; with these buffers, the pH of the starring suspension ranged from 7.3-7.6. Since the desired pH is 7.3-7.4, all the following test series with cholate as surfactant were done with buffer pH 7.1)

Insulin Solution A:
4 mg/mL, 8 mg/mL, 10 mg/mL, 20 mg/mL phosphate buffer, pH 7.4
30 μL HCl (1M) per mL dissolved dry insulin,
followed by 30 μL 1 M NaOH per 1 mL solution

Insulin Solution B:
4 mg Actrapid/mL phosphate buffer, pH 7.4
   Insulin-vesicle mixtures
5 w-% total lipid concentration
0.04, 0.08, 0.1 and 0.2 mg dry insulin per 1 mg total lipid
(4, 8, 10, 20 rel. w-%)

Preparation of vesicle suspension was done as described in examples 1-27, using similar membrane composition. However, to achieve high insulin/lipid ratios using reasonably high final total lipid concentrations dry insulin was dissolved to the concentration higher than that used in commercial solutions.

Lyophilised human recombinant insulin does not dissolve readily in phosphate buffer with pH 7.4. To prepare insulin solution, dry, lyophilised human recombinant insulin “powder”, analogous to Actrapid™, was therefore first added to 2 mL buffer and vortexed thoroughly. After a transient acidification (achieved by the addition of 60 μL HCl), which increased insulin solubility sufficiently to give rise to a clear solution, 60 μL NaOH was added to adjust pH back to 7.4, where insulin is stable (as hexamers) and resistant to degradation/desamidation. An additional solution was prepared by directly dissolving 8 mg insulin in 2 mL buffer, pH 7.4.

Vesicles suspension (2 mL) and insulin solution-A (2 mL) were mixed thoroughly and incubated for 12 hours at the above given nominal insulin/lipid ratios. The final total lipid concentration was 50 mg/mL in all cases. For reference, solution B was used. The rest of experiment was performed as described in examples 1-27.

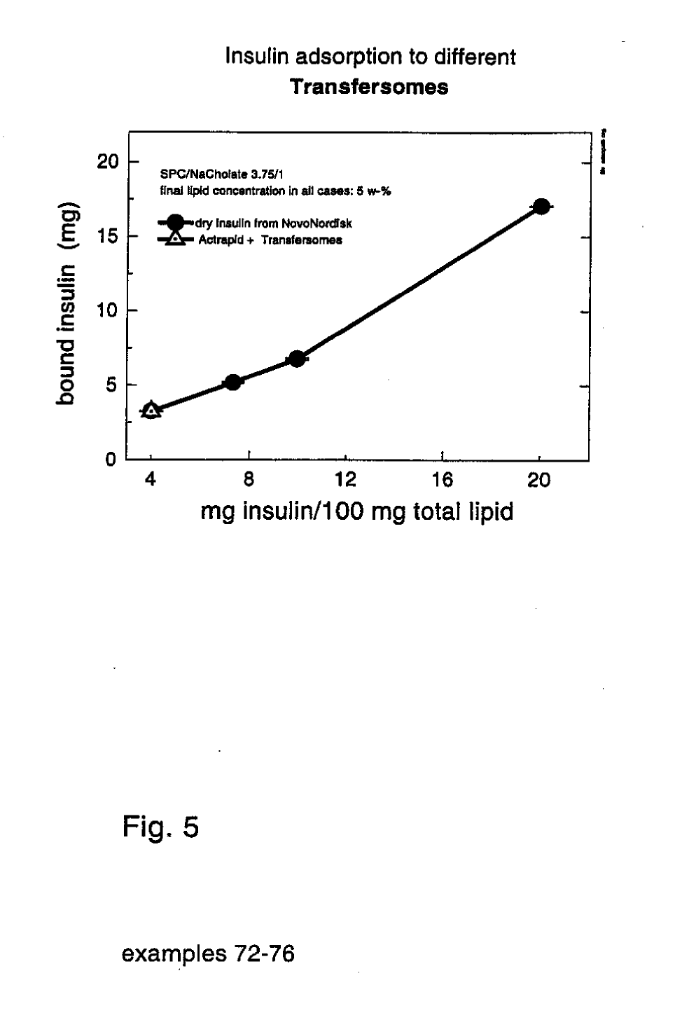

Results. Insulin binding from the solution made from the dry protein powder (which at least temporarily gives raise to monomer solution) is comparable to that measured with insulin from Actrapid in examples 1-27 (FIG. 5). This suggests that it is possible to associate a high amount of insulin with the suspension of lipid vesicles at concentration 50 mg/mL. Insulin binding maximum is found around protein/lipid weight ratio of 1/5, where approximately 16 mg of the added insulin associate with the mixed lipid membranes.

At similar protein concentration, identical results are measured with the ad hoc dissolved and commercial insulin solutions.

In the following experimental series, the adsorption of insulin to different charged and uncharged, fluid, mixed lipid membranes was compared.

### Examples 77-92

Conventional vesicles, SPC liposomes, neutral (TL=10 w-%):
no net charge, comprising only zwitterionic phospholipids
1 g phosphatidylcholine from soy-bean
9 mL phosphate buffer, pH 7.4

Conventional vesicles, charged SPC/SPG liposomes (TL=10 w-%):
net negative charge from 25 mol-% anionic phosphatidylglycerol
750 mg phosphatidylcholine from soy-bean
250 mg phosphatidyglycerol from soy-bean
9 mL phosphate buffer, pH 7.4

Highly deformable neutral vesicles (TL=10 w-%):
no net charge, comprising zwitterionic phospholipids and non-ionic surfactants
550 mg phosphatidylcholine from soy-bean
450 mg Tween 80
9 mL phosphate buffer, pH 7.4

Highly deformable charged vesicles A (TL=10 w-%):
net negative charge, due to 25 mol-% anionic cholate
874.4 mg phosphatidylcholine from soy-bean
125.6 mg sodium cholate
9 mL phosphate buffer, pH 7.1

Highly deformable charged vesicles B (TL=10 w-%):
net negative charge, due to 25 mol-% (rel. to PC) of anionic phosphatidyiglycerol
284.3 mg phosphatidylcholine from soy-bean
94.8 mg phosphatidylglycerol from soy-bean
620.9 mg Tween 80
9 mL phosphate buffer, pH 7.4

Insulin-vesicle mixtures, respectively
50, 25, 10, 5 mg total lipid per mL final suspension
0.04, 0.08, 0.1 and 0.2 mg insulin per 1 mg total lipid
(4, 8, 10, 20 rel. w-% of protein)

All vesicles were prepared as described before. Tween-containing vesicles were stirred for 7 days. The cholate-containing vesicles and liposomes were stirred for 2 days. Actrapid 100 HM™ (Novo-Nordisk) was the source of insulin. This caused the final protein and the resulting final lipid concentration to vary (50, 25, 10 and 5 mg TL/mL, respectively). With SPC-liposomes, however, only 4 rel. w-% sample was investigated.

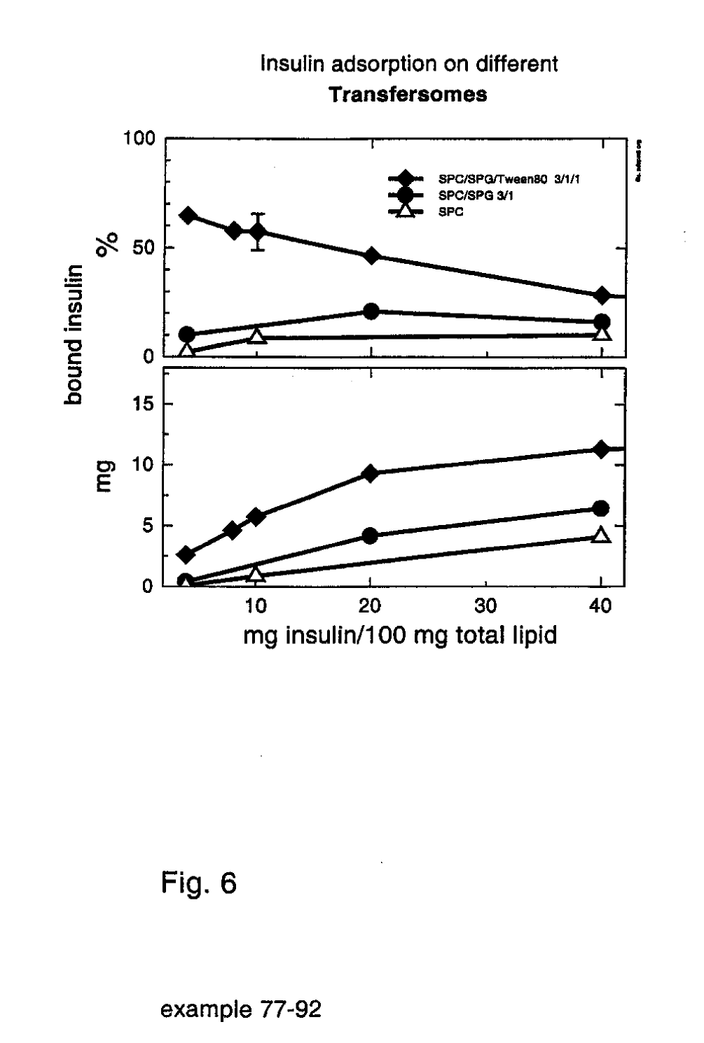

Experimental protocol is the same as described for examples 1-27. The incubation time was 3 hours, the centrifugation time was 6 hours (at 500 g) for all preparations to make comparisons easier. The results of measurements are shown in FIG. 6.

Results clearly show that insulin, despite its net negative charge, binds best to the negatively charged surfaces. High membrane flexibility, which permits high vesicle deformability, is also advantageous.

Relative binding efficiency is 80-90% for the highly flexible, charged membranes. Such, very high, degree of protein membrane association is observed at 1/25 insulin/lipid weight ratio for both types of investigated phospholipid-surfactant mixtures. Uncharged membranes comprising phospholipids and nonionic surfactants show 50% relative binding at comparable insulin/lipid ratios. However, only between 2.5% (cf. experiments 28-45) added insulin is calculated to bind to the uncharged, phosphatidylcholine liposomes. This, worst of all, result is surpassed by protein binding to the charged liposomes, which associate with 10-20% of added insulin at the protein/lipid weight ratio of 1/25. Charged conventional lipid bilayers are hence intermediate between uncharged liposomal membranes and the more flexible but neutral (Transfersome™) membranes.

Such findings suggest that net surface charges (originating from charged lipids or other charged membrane-associated components) should be combined with membrane softness (which is promoted by the existence of detergents and other related molecules in the adsorbent) to maximise surface or carrier-protein association. It stands to reason that the charges “pull” (parts of) adsorbing molecules to the adsorbent which, when “softened” permits an easy insertion of the protein into interfacial region.

### Example 93-95

Conventional vesicles, SPC liposomes, neutral (TL=10 w-%):
no net charge, comprising only zwitterionic phospholipids
1 g phosphatidylcholine from soy-bean
9 mL phosphate buffer, pH 7.4

Highly deformable charged vesicles A (TL=10 w-%):
net negative charge, due to 25 mol-% anionic chelate
874.4 mg phosphatidylcholine from soy-bean
125.6 mg sodium chelate
9 mL phosphate buffer, pH 7.1

Highly deformable charged vesicles B (TL=10 w-%):
net negative charge, due to 25 mol-% (rel to PC) of anionic phosphatidylglycerol
284.3 mg phosphatidylcholine from soy-bean
94.8 mg phosphatidylglycerol from soy-bean
620.9 mg Tween 80
9 mL phosphate buffer, pH 7.4

Insulin-vesicle mixtures, respectively
50, 25, 10, 5 mg total lipid per mL final suspension
0.04, 0.08, 0.1 and 0.2 mg insulin per 1 mg total lipid
(4, 8, 10, 20 rel. w-% of protein)

Preparation. To study the kinetics of insulin adsorption: to phosphatidylcholine Tween 80 mixed membranes, we made time dependent measurements. Test vesicles were prepared as described in the corresponding previous examples. The first data points were taken 2 hours after mixing the lipid suspension with protein solution. For the neutral highly deformable membranes, the next time point was chosen to be 3 hours. Further samples, for all suspensions, were taken after 4 or 5 days and after 5 or 6 weeks of incubation.

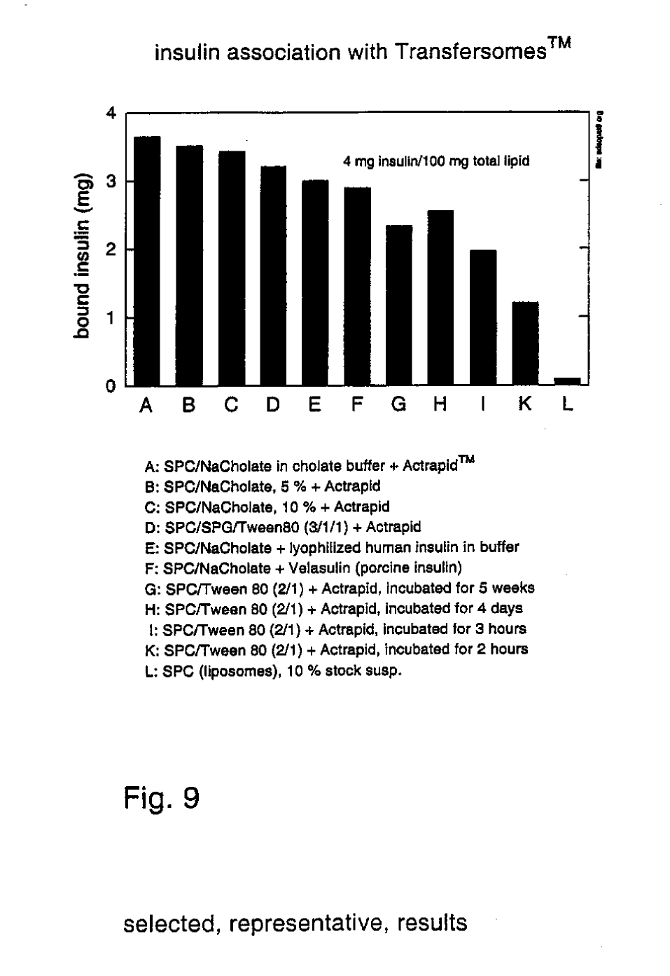

Results. A clear time dependency was discovered for adsorption of insulin to uncharged SPC/Tween mixed membranes (see FIG. 9 for some representative data). Binding efficiency observed early during the association process increased from 30%, at 2 hours, to 50%, at 3 hours, when nominal insulin/lipid weight ratio was 1/25. At t=4 days, the binding increased to 64%, but this difference may be insignificant as after 5 weeks the binding was only 58%.

The binding of insulin to simple phosphatidylcholine liposomes was measured to increase only marginally from 2.5% after 3 hours to 5% after 6 weeks.

Insulin adsorption to the charged SPC/SPG/Tween 80 mixtures is much faster and stronger than in the case of neutral membranes, as indicated by an increase in protein binding to such membranes, from 64% after 2 hours to 76% after 6 weeks. The smallness of secondary increase, compared to the magnitude of first hours association, is indicative of a rather fast binding kinetic.

The rate of insulin binding is even higher for the charged SPC/cholate mixed membranes. Experiments done with such charged vesicles reveal no time dependence of protein adsorption to the mixed lipid membrane. At 2 hours, the binding is already as complete as after 5 weeks of incubation, within the experimental error. This suggests that insulin adsorption to charged, flexible membranes is much faster than to the non-charged membranes. By inference, we suggest that non-trivial electrostatic interactions also might affect the desorption of protein molecules. The very weak and/or slow insulin association with phosphatidylcholine membranes shows that hydrophobic binding alone is insufficient for achieving high payloads. This may be due to the limited capability of insulin molecules to find suitable binding places at the lipid bilayer surface. Repulsion between the few, inconveniently, adsorbed protein molecules and the next tentative adsorbates could be important as well.

### Examples 96-100

Suspensions of ultradeformable vesicles with different charge density (TL=10 w-%):
net negative charge, due to 25, 35, 50, 67, 75 mol-% phosphatidylglycerol
137 mg, 205 mg, 274 mg, 343 mg, 411 mg phosphatidylglycerol from soy-bean
411 mg, 343 mg, 274 mg, 205 mg, 137 mg phosphatidylcholine from soy-bean
452 mg Tween 80
9 mL phosphate buffer, pH 7.4
2 mg insulin/mL final suspension

Lipid vesicles were prepared as described in examples 93-95. Increasing relative concentration of charged lipid in the membrane enhanced vesicle-insulin association, as is seen in FIG. 4, and moderately but acceptably enlarged the viscosity of final suspension.

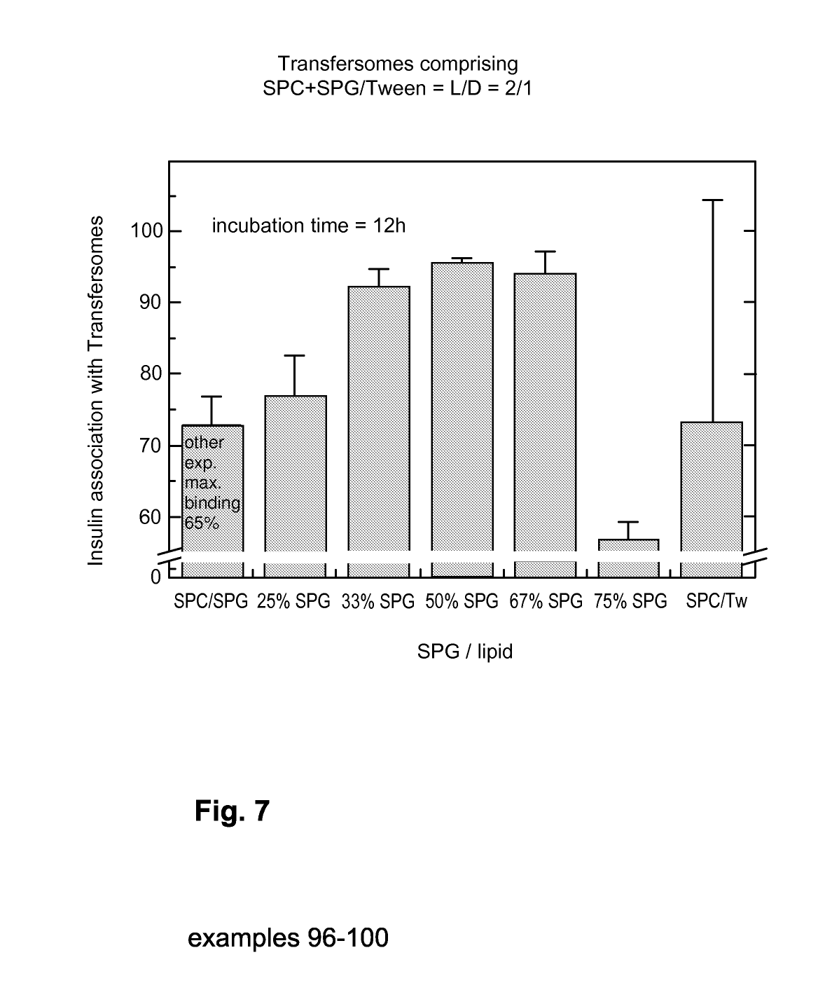

The lipid suspension at the higher SPG/SPC molar ratios, prepared as in examples 93-95, were rather viscous and difficult to handle. Higher relative concentration of the charged lipid component did increase relative amount of vesicle associated insulin, however. FIG. 7 illustrates this.

Changing charged lipid content affects the efficiency of protein (insulin) binding in a non-monotonous fashion. At first, the relative amount of vesicle-associated insulin increases. At SPC/SPG ratio close to 50, maximum relative binding is observed. This suggests that very high SPG content is detrimental to efficient insulin binding, possibly is owing to the interracial crowding effect and/or to the influence of surface charges on protein adsorption kinetics. (The latter should not be too fast to permit macromolecular rearrangements at the surface and thus lead to maximum packing density.)

### Examples 101-104

Highly flexible charged membranes (TL=:10 w-%) mixed 1/1 with insulin
874.4 mg phosphatidylcholine from soy-bean
125.6 mg sodium cholate
9 mL phosphate buffer, pH 7.1
4 mg insulin/mL in starting solution

Different methods were used for vesicle preparation: in addition to the extrusion and freeze-thaw cycles, described in examples 1-27, a much simpler protocol (in which the suspension is only extruded sequentially) was also tested.

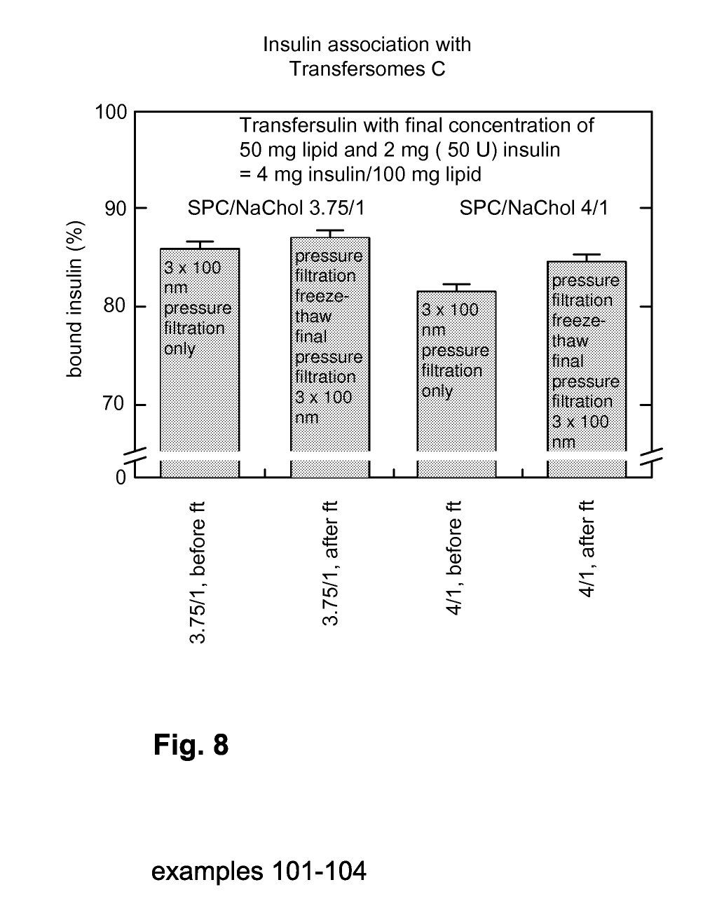

No significant difference in the efficiency of protein adsorption to the mixed lipid membranes was found (FIG. 8). However, the shape adaptability of lipid vesicles, as assessed in “confining pore penetration assay”, was different for the different batches: the deformability of vesicle prepared as in examples 1-27 was found to be the highest.

### Examples 105-106

Ultraflexible charged membranes with various additives (final composition)
437 mg phosphatidylcholine from soy-bean
63 mg sodium cholate
1 mL phosphate buffer, pH 7.1
2 mg insulin/mL in final suspension

Additive A
m-cresol 1.5 mg/mL (final)

Additive B
benzyl alcohol 2.5 mg/mL (final)

Co-solvent addition to the Transfersomes® containing sodium cholate affects the final membrane-associated insulin amount. Relative efficiency of binding is 60% in the presence of m-cresol and 90% after the introduction of benzyl alcohol into test suspension.

The additives used in examples 103-104 also can act as preservatives.

### Examples 107-110

Similar membranes with different insulin from different sources
437 mg phosphatidylcholine from soy-bean
63 mg sodium cholate
1 mL phosphate buffer, pH 7.1
2 mg insulin/mL
   from Actrapid 100 HM™ (Novo-Nordisk)
   originally dry, human recombinant (Novo-Nordisk)
   originally dry, porcine (Sigma Chemical Industries)
   from Lispro™, an insulin analogue (Pfizer Inc.)

No significant differences were observed in the efficiency of different protein adsorption to similar membranes. This does not exclude the possibility of different rates of de/adsorption, however.

In particular, dry insulin, if dissolved in an acidic buffer and brought back into the neutral pH range, adsorbs to the mixed lipid membranes as efficiently as insulin from a ready to use Actrapid™ (Novo-Nordisk) solution.

### Examples 111-118

Soft uncharged membranes
Starting suspension (10% TL):
1099.7 mg phosphatidylcholine from soy-bean
900.3 mg Tween 80
19 mL phosphate buffer, pH 7

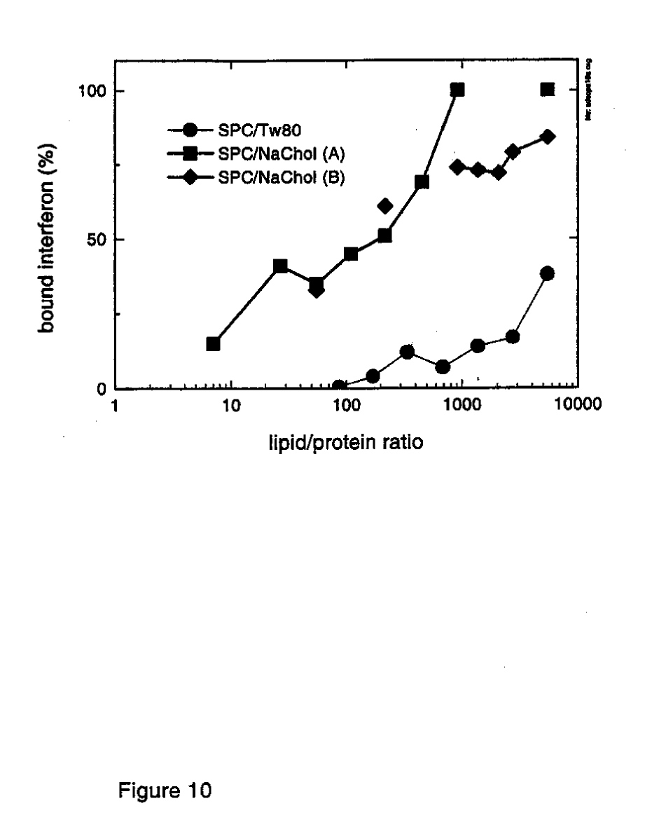

Final suspension comprising:
8.4 μg IF mixed with the lipids blend as given above,
using 1.84 mg TL/mL to 18.4 μg TL/mL to create
increasing relative amounts of interferon, as given in FIG. 10

Formulations contained protein/lipid mixtures with increasing molar ratio and were prepared essentially as described in examples 60-71. The tests were done as described in examples 1-27 with two modifications. The first involved the dealing with Centrisart separation tubes (cut-off 100 kDa), which in this test series were always pre-coated with albumin (from a solution containing 40 mg BSA/mL buffer) to decrease the level of non-specific protein adsorption below 15%. After incubation with BSA, the tubes were therefore washed twice with the buffer and filled with interferon solution of appropriate concentration (prepared by diluting the stock solution in the same buffer). To assess final protein concentration, commercial ELISA immunoassay for IF was used. To calculate the amount of vesicle-associated interferon the same procedure as is described with examples 1-18 was used. The degree of protein binding was thus identified with the “loss of protein” from the supernatant, measured in duplicate or triplicate.

The results are given in FIG. 10. They reveal a picture qualitatively similar to that described for insulin binding.

### Examples 119-134

Highly flexible, charged membranes
Starting suspension
Total lipid (TL) content 10 w-% comprising:
   874.4 mg phosphatidylcholine from soy-bean
   125.6 mg sodium cholate
   9 mL phosphate buffer, pH 7.1
Final suspensions
   lipid/protein mixtures as given in FIG. 10
   (other data corresponding to those given with examples 111-118)

The results of two different experimental series, illustrated in FIG. 10 (filled diamonds and squares), indicate the desirable action of negative membrane charge on the efficiency of interferon binding to the highly deformable bilayers, despite the net negative charge on protein molecules.

### Examples 135-145

Starting suspension (10% TL):

Soft, uncharged membranes
SPC/Tw80
550 mg phosphatidylcholine from soy-bean
450 mg Tween 80
9 mL phosphate buffer, pH 6.5

Soft, charged membranes
SPC/NaChol
874.4 mg phosphatidylcholine from soy-bean
125.6 mg sodium cholate
9 mL phosphate buffer, pH 7.1

Final suspension comprising:
lipids in the ratios given above and
10000 IU of interleukin-2 (IL-2)

The given lipid mixture and proteins were processed together. Then the degree of association was determined. The separation was done essentially as described for examples 119-134 whereas the amount of IL-2 was determined using the protein dependent stimulation of Renca-cells growth in vitro, compared to a standard curve. This yielded the data given in following table. (Absolute IL-2 concentrations are given in IU and relative protein amounts in %):

Efficiency of interleukin association with ultradeformable
vesicle as a function of time

| | day 1 | | | | day 6 | | | |
|---|---|---|---|---|---|---|---|---|
| | SPC/NaChol | | SPC/Tw80 | | SPC/NaChol | | SPC/Tw80 | |
| | IU | % | IU | % | IU | % | IU | % |
| Starting | 10000 | 69 | 10000 | 190 | 10000 | 154 | 10000 | 364 |
| Bound | 8000 | 55 | 1000 | 19 | 5750 | 88 | 750 | 27 |
| Free | 6500 | 45 | 4250 | 81 | 750 | 12 | 2000 | 73 |
| Recovered | 14500 | 100 | 5250 | 100 | 6500 | 100 | 2750 | 100 |

Deviations between the starting and final (total recovered protein) values are partly due to the loss of protein during vesicle/IL-2 separation, and partly to modified protein activity by the presence of lipids.

Short term association of interleukin and pre-formed highly deformable lipid vesicles with different surface charge density was found to be less sensitive to the charge effect than suggested by above table (data not shown).

### Examples 146-148

Conventional neutral vesicles (starting suspension):
1 g phosphatidylcholine from soy-bean
9 mM phosphate buffer, pH 6.5

Highly deformable neutral vesicles (starting)
550 mg phosphatidylcholine from soy-bean
450 mg Tween 80
9 mL phosphate buffer, pH 6.5

Highly deformable charged vesicles (starting):
874.4 mg phosphatidylcholine from soy-bean
1%5.6 mg sodium cholate
9 mL phosphate buffer, pH 7.1

Calcitonin-(ex. salmon) mixed with vesicles (final suspension)
100 mg total lipid per mL final suspension
1 mg protein per 100 mg total lipid

All lipid suspensions were prepared as described before. The protein (spiked with a small amount of ¹²⁵I-labelled protein, purified shortly before use) was added to the preformed vesicles and incubated for at least 24 hours; alternatively, the protein solution was added to the lipids and co-extruded through the micro-porous filter during suspension preparation.

To determine the relative efficiency of polypeptide binding to the membranes, the protein/vesicle mixture was chromatographed using size-exclusion gel chromatography with subsequent radioactivity detection. This afforded two peaks that contained radiolabelled protein, associated with the vesicle and in the solution, respectively. The areas under the curve were around 30% and 70% for conventional vesicles, at 60-70% and 40-30% for the neutral, soft membranes and > 80% and < 20% for the charged, highly flexible membranes, respectively.

### Examples 149-152

Highly deformable neutral vesicles (starting)
550 mg phosphatidylcholine from soy-bean
450 mg Tween 80
9 mL phosphate buffer, pH 6.5

Highly deformable charged vesicles (starting):
874.4 mg phosphatidylcholine from soy-bean
125.6 mg sodium cholate
9 mL phosphate buffer, pH 7.1

Immunoglobulin G mixed with vesicles (final suspension)
100 mg total lipid per mL final suspension
0.5 mg and 1 mg protein per 100 mg total lipid

All lipid suspensions were prepared as described before. The immunoglobulin (a monoclonal IgG directed against fluorescein) was incorporated in the formulation by the addition into preformed vesicle suspension. After the separation of vesicle associated and free immunoglobulin amounts, the relative contribution from the former was determined by using fluorescence quenching in the separated, original, and control solutions. This afforded the final IgG concentration in each compartment.

The efficiency of IgG carrier membrane association was estimated to be at least 85% in the case of charged, highly deformed vesicles and app. 10% lower for the neutral, soft membranes. The smallness of observed difference is probably due to the fact that Ig contains a large hydrophobic Fc region, which inserts readily into the lipid membrane even in the absence of membrane softening, defects generating components.

### Examples 153-154

Highly deformable charged vesicles, Type C:
130.5 mg phosphatidylcholine from soy-bean
19.5 mg cholate, sodium salt
0.1 mL ethanol

Highly deformable uncharged vesicles, Type T:
75 mg phosphatidylcholine from soy-bean
75 mg Tween 80
0.1 mL ethanol

Insulin, human recombinant:
1.35 mL Actrapid™ 100 (Novo-Nordisk)

Test formulation. Either lipid mixture was taken up in alcohol, until a uniform phospholipid solution was obtained (Cave: Na cholate does not dissolve perfectly!). The mixture was injected into an insulin solution and mixed thoroughly. After ageing for approximately 12 h, the resulting suspension of “crude vesicles” was filtered several times through a O₂ micrometer filter (Sartorius, Göttingen), in order to facilitate, and achieve, good sample homogeneity. The final insulin concentration was 80 IU/mL.

Test. A healthy male volunteer (75 kg, age 42) fasted for 17 hours prior to the first glucose concentration determination. To follow the temporal variation of glucose concentration in his blood, 2 mL to 4 mL samples were drawn every 10 min to 20 min through a soft intravenous catheter placed in one arm. After an initial test period of 70 min, during which the average blood glucose concentration was 78.4, the type C Transfersulin® suspension was applied (45 IU) and uniformly smeared over the intact skin surface on the inner side of the other forearm (in several sequences) so as to cover an area of 56 cm². 30 minutes after the application of test suspension, the skin surface appeared macroscopically dry; 30 minutes later, only faint traces of the suspension were visible.

A standard glucose-dehydrogenase assay (Merck, Gluc-DH) was used to determine the blood sugar concentration. Each specimen contained three independent samples and each measurement was made at least in triplicate. This ensured the standard deviation of the mean seldom to exceed 5 mg/dL, typical error being of the order of 3 mg/dL.

Results. The change of blood glucose concentration in a normoglycaemic volunteer test person after an epicutaneous administration of insulin associated with Transfersomes® (Transfersulin®) was always slower man that achieved by a subcutaneous injection of an insulin solution.

Maximum decrease of glucose concentration in the blood after an epicutaneous administration of Transfersulin® typically exceeded 10% of that resulting from the corresponding subcutaneous injection, the area under the curve being 20%, at least, using published data as a reference. The average suppression of blood glucose concentration in the blood in the case of suspension C for t> 3 h amounted to approx, −18 mg/dL.

The result for suspension T was approximately 35% inferior to the data measured with suspension C. Incorporation of phosphatidylglycerol (15 w-% relatively to phosphatidylcholine) reduced the difference between C- and T-type formulations to 25% (data not shown).

However, even the best other noninvasive insulin delivery methods available to date, such as the use of iontophoresis (Meyer, B. R., Katzeff, H. L., Eschbach, J., Trimmer, J., Zacharias, S. R., Rosen, S., Sibalis D. Amer. J. Med. Set 1989, 297: 321-325) or transnasal sprays bring less than 5% and less than 10% of the insulin molecules, respectively, into the systemic blood circulation.

### Example 155

Highly deformable charged vesicles:
composition as in examples 72-76.

Insulin, human recombinant:
Actrapid™ (lyophilisate) as in examples 72-76 (Novo-Nordisk)

Test formulation was prepared as described in examples 61-65. Administration was done essentially as described in the previous examples, but the fasting period lasted longer and the blood sampling begun earlier. (The experiment thus begun with 12 hours of non-monitored fasting, a further fasting period of 12 h, during which the blood glucose level was monitored without any treatment, and a monitored period of 16 h during which the test person fasted and was treated with epicutaneous Transfersulin®. Further difference was that the application area was only 10 cm².

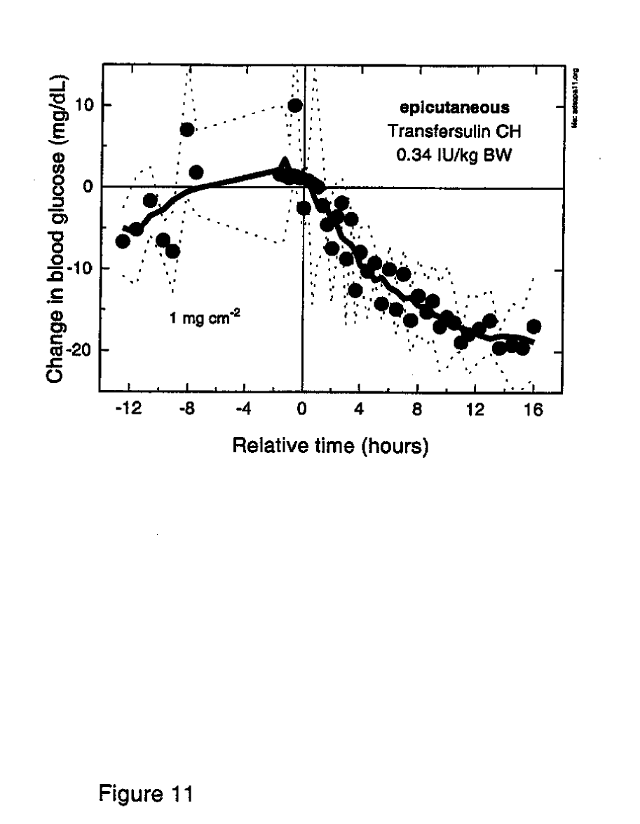

Before the administration of insulin, samples were taken at irregular times. After Transfersulin® administration, the blood samples were drawn every 20 min over the first 4 hours and every 30 min therafter. All samples were analysed with Accutrend (Boehringer-Mannheim, Germany), a self-diagnosis device. Three to five readings were taken at every time point. The results given in FIG. 11 correspond to the mean value of the blood glucose concentration change. Dashed lines give 95% confidence limits.

In the second “no-treatment” period the average blood glucose concentration was 83.2 mg/dL. Lowering of the blood glucose concentration within the first hours following epicutaneous drug administration by means of highly adaptable mixed lipid vesicles is clearly seen. Glucodynamic profile is similar to that measured in previous test series, the overall effect being somewhat stronger, probably due to the much higher drug concentration in the latter test formulation.

### Examples 156-158

Highly deformable charged vesicles:
composition as in example 153.

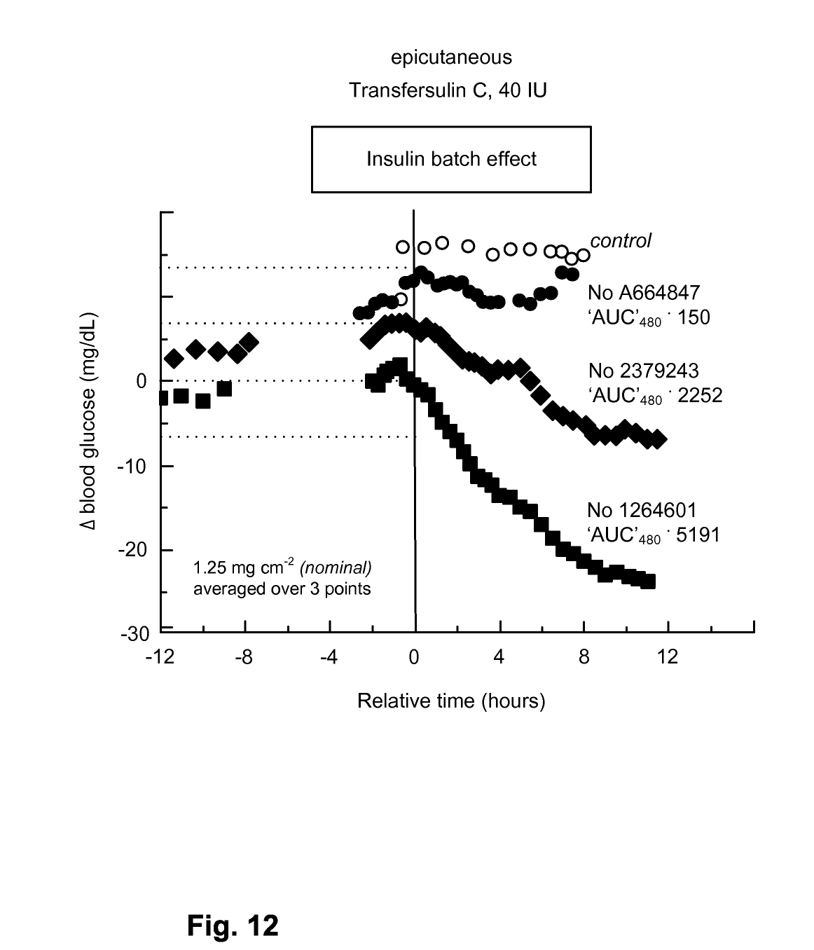

Insulin, human recombinant:
Actrapid™ (Novo-Nordisk), batches as given in the FIG. 12.

In this test series, the effect of inter-batch variability for insulin was studied, by using the same Transfersome® batch. Administration was done as described in the previous examples. The dose per area also was similar to that use in previous examples.

The average blood glucose concentration was approximately the same in all three experiments. This notwithstanding, the outcome of experiments was vastly different between the insulin batches. One batch worked very well and one not at all; the third lot produced intermediate results.

Small batch-to-batch variabilities for the insulin (which are known, but usually not reported, and are particularly prominent in the presence of very large adsorbing (carrier) surface, seem to affect the efficiency and/or the kinetics of insulin-carrier interaction. Changed rate of drug liberation is believed to be particularly sensitive to the phenomenon. It is therefore important not only to study the amount of carrier associated lipid prior to serious biological tests but, moreover, to determine the rate of drug liberation. Measuring glucodynamics in the test animals, such as mice or rats, as a formulation characteristics after an injection is useful for this purpose.

Glucodynamics in a normoglycaemic human volunteer after the administration of three different Transfersulin® batches with identical Transfersomes® but different insulin batches clearly the relatively strong effect of even small changes in the original drug characteristics on the biological activity of final formulation (see FIG. 12).

### REFERENCES

Cevc, G., Strohmaier, L., Berkholz, J., Blume, G. Stud, Biophys. 1990, 138: 57ff
Cevc, G., Hauser, M., Kornyshev, A. A. Langmuir 1995, 11: 3103-3110.
Prime, K. and Whitesides, G. M. Science, 1991, 252: 1164-1167
Deber, C. M.; Hughes, D. W.; Fraser, P. E.; Pawagi, A. B.; Moscarello, M. A. Arch Biochem. Biophys. 1986, 245: 455-463.

Zimmerman, R. M., Schmidt, C. F., Gaub, N. H. E. J. Colloid Int. Sci. 1990, 139: 268-280.
Hernandez-Caselles, T.; Villaiaain, J.; Gomez-Fernandez, J. C. Mol. Cell. Biochem. 1993, 120: 119-126.
Scott, D. L.; Mandel, A. M.; Sigler, P. B.; Honig, B. Biophys. J. 1994, 67: 493-504.
Norde, W., in Adv. Colloid Interface Sci. 1986, 25: 267-340.
Lee, C. S.; Belfort, G. Proc. Natl. Acad. Sci., 1989, 86: 8392-8396.
Haynes, C. A.; Norde, W. Colloids and Surfaces B 1994, 2, 517ff.
Haynes, C. A.; Sliwinski, E.; Norde, W. J. Colloid Interface Sci. 1994, 164, 394ff.
Proteins at Interfaces, T. A. Horbett and J. L. Brash, eds., ACS Symposium Series 602, 1995, New York.
Torchilin, V. P.; Goldmacher, V. S.; Smirnov, V. N. Biochem. Biophys. Res. Comm. 1978, 85: 983-990.
Meyer, B. R., Katzeff, H. L., Eschbach, J., Trimmer, J., Zacharias, S. R., Rosen, S., Sibalis, D. Amer. J. Med. Sci. 1989, 297: 321-325.

### Other Informative Literature

### Patents

Pauly, M.; Koulbanis, C. Liposomes containing amino acids and peptides and proteins for skin care. FR/Patent # 2627385/89.
Loughrey, H. C.; Cullis, P. R.; Bally, M. B.; Choi, L. S. L.; Wong. K. F. Targeted liposomes and methods using derivatized lipids for liposome-protein coupling. PCT#9100289/91.
Hostetler, K. Y.; Feigner, P. L.; Felgner, J. Liposomes for prolonging the bioavailability and shelf life of therapeutic peptides and proteins. PCT # 9104019/91
Matsuda, H.; Ueda, Y.; Yamauchi, K.; Inui, J. Sustained-release protein-liposome complexes. JP # 0482839/92
Kobayashi, N.; Ishida, S.; Kumazawa, E. Method of quantitating liposome-encapsulated bioactive proteins. JP # 05302925/93
Tagawa, T.; Hosokawa, S.; Nagaike, K. Drug-containing protein-bounded liposome. EPT# 526700/93.

### Protein-Liposome Interactions

Ledoan, T.; Elhajji, M.; Rebuffat, S.; Rajesvari, M. R.; Bodo, B. Fluorescence studies of the interaction of trichorianine a 3c with model membranes. Biochim. Biophys. Acta 1986, 858: 1-5.
Krishnaswamy, S. Prothrombinase complex assembly contributions of protein-protein and protein-membrane interactions toward complex formation. J. Biol. Chem. 1990, 265: 3708-3718.
Liu, D.; Huang, L. Trypsin-induced lysis of lipid vesicles: effect of surface charge and lipid composition. Anal. Biochem. 1992, 202: 1-5.

1. A pharmaceutical composition comprising:
(i) a bilayer vesicle consisting essentially of:
at least one phosphatidylcholine; and
a nonionic detergent, at least one detergent;
(ii) at least one macromolecule; and
(iii) an aqueous liquid medium, wherein the macromolecule resides on the surface of the vesicle.

2. The composition of claim 1, wherein, prior to association of the macromolecule with the surface of the vesicle, the surface of the vesicle carries a net electric charge and the macromolecule carries a net electric charge, and the net electric charge of the surface of the vesicle and the net electric charge of the macromolecule have the same sign.

3. The composition of claim 2, wherein the surface of the vesicle is negatively charged and wherein the macromolecule is negatively charged.

4. The composition of claim 75, wherein the nonionic detergent is a long-chain fatty acid; a long-chain fatty alcohol; an alkyl-N-methylglucamide; an alkanoyl-N-methylglucamide; a polyethylene-glycol-octylphenyl ether; nonaethylenglycol-octylphenyl ether; a polyethylene-acyl ether; a nonaethylen-dodecyl ether; a polyethylenegylcol-isoacyl ether; an octaethyleneglycol-isotridencyl ether; a polyethylene-acyl ether; an octaethylenedodecyl ether; a polythyleneglycol-sorbitane-acyl ester; polyethyleneglycol-20-monolaurate; polyethyleneglycol-20-sorbitan-monooleate; a polyhydroxyethylene-acyl ether; a polyhydroxyethylene-lauryl ether; a polyhydroxyethylenemyristoyl ether; a polyhydroxyethylene-cetylstearyl ether; apolyhydroxyethylene-oleoyl ether; a polyhydroxyethylene-4, or 6, or 8, or 10, or 12-lauryl ether; a Brij; Myrj 45; a polyhydroxyethylene-laurate; a polyhydroxyethylene-oleate; polyethoxylated castor oil 40; a sorbitan-monoalkylate; sorbitane-monolaurate; an acyl-N-methylglucaraide; an alkanoyl-N-methylglucamide; decanoyl-N-methylglucamide; dodecanoyl-N-methylglucamide; or a surface-active polypeptide.

5. The composition of claim 75, wherein the nonionic detergent is a polyethyleneglycol-sorbitan-acyl ester, a Brij, a Myrj, a polyhydroxyethylene-8-stearate, a polyhydroxyethylene lauryl ether, a polyethylene-glycol-octylphenyl ether, a nonaethylene-glycol-octylphenyl ether, Triton, or an —N-glucamide or a sorbitan monoalkylate nonionic detergent.

6. The composition of claim 1, wherein the macromolecule is a hormone.

7. The composition of claim 1, wherein the macromolecule is human recombinant insulin or humanized insulin.

8. The composition of claim 1, wherein the macromolecule is interleukin suitable for the use in humans or animals and is IL-2, IL-4, IL-8, IL-10, or IL-12.

9. The composition of claim 1, wherein the macromolecule is interferon alpha, beta or gamma.

10. The composition of claim 1, wherein the macromolecule is immunoglobulin (Ig) IgA, IgG, IgE, IgD or IgM.

11. The composition of claim 1, wherein the macromolecule is calcitonin.

12. (canceled)

13. The composition of claim 1, wherein the phosphatidylcholine is from soy-bean.

14. The composition of claim 75, wherein the nonionic detergent is a polyethyleneglycol-sorbitan-acyl ester.

15. The composition of claim 14, wherein the polyethyleneglycol-sorbitan-acyl ester is polyethyleneglycol-20-sorbitan-monooleate or polyethyleneglycol-20-sorbitan-monolaurate.

16. The composition of claim 75, wherein the nonionic detergent is a Brij.

17. The composition of claim 75, wherein the nonionic detergent is a Myrj.

18. The composition of claim 17, wherein the Myrj nonionic detergent is Myrj-45.

19. The composition of claim 1, wherein the macrormolecule is a protein.

20. The composition of claim 1, wherein the macrormolecule is a polynucleotide.

21. The composition of claim 1, wherein the macrormolecule is a polysaccharide.

22. The composition of claim 19, wherein the protein is an enzyme, an interferon, an interleukin, an immunoglobulin, or a hormone.

23. The composition of claim 19, wherein the protein is insulin.

24-28. (canceled)

29. The pharmaceutical composition of claim 1, wherein the detergent is sodium cholate.

30. The composition of claim 29, wherein, prior to association of the macromolecule with the surface of the vesicle, the surface of the vesicle carries a net electric charge and wherein the macromolecule carries a net electric charge, and the net electric charge of the surface of the vesicle and the net electric charge of the macromolecule have the same sign.

31. The composition of claim 30, wherein the surface of the vesicle is negatively charged and wherein the macromolecule is negatively charged.

32. The composition of claim 29, wherein the macromolecule is a hormone.

33. The composition of claim 32, wherein the hormone is human recombinant insulin or humanized insulin.

34. The composition of claim 29, wherein the macromolecule is interleukin suitable for the use in humans or animals and is IL-2, IL-4, IL-8, IL-10, or IL-12.

35. The composition of claim 29, wherein the macromolecule is interferon alpha, beta or gamma.

36. The composition of claim 29, wherein the macromolecule is immunoglobulin (Ig) IgA, IgG, IgE, IgD or IgM.

37. The composition of claim 29, wherein the macromolecule is calcitonin.

38. The composition of claim 29, wherein the phosphatidylcholine is from soy-bean.

39. The composition of claim 29, wherein the macromolecule is a protein.

40. The composition of claim 39, wherein the protein is an enzyme, an interferon, an interleukin, an immunoglobulin, or a hormone.

41. The composition of claim 40, wherein the hormone is insulin.

42. The composition of claim 29, wherein the macromolecule is a polynucleotide.

43. The composition of claim 29, wherein the macromolecule is a polysaccharide.

44-74. (canceled)

75. The composition of claim 1, wherein the detergent is a nonionic detergent.

76. The composition of claim 1, wherein the detergent is an ionic detergent.

77. The composition of claim 76, wherein the ionic detergent is an alkyl-trimethyl-ammonium salt; an alkyl-dimethyl-ammonium salt; an alkylsulfate salt; a monovalent salt of cholate; a monovalent salt of deoxycholate; a monovalent salt of glycocholate; a monovalent salt of glyeodeoxycholate; a monovalent salt of taurodeoxycholate; a monovalent salt of taurocholate; an acyl-dimethyl-aminoxide; an alkanoyl-dimethyl-aminoxide; a dodecyl-dimethylaminoxide; N-alkyl-N,N-dimethylglycine; 3-(acyldimethylammonio)-alkanesulfonate; N-acyl-sulfobetaine; lauryl sulfate; oleoyl sulfate, sodium deoxycholate; sodium glyeodeoxycholate; sodium oleate; sodium taurate; a fatty acid salt; sodium eiaidate; sodium linoleate; sodium laurate; a lysophospholipid; n-octadecylene-glycerophosphatidic acid; n-octadecylene-phosphorylglycerol; n-octadecylene-phosphorylserine; n-alkyl-glycerophosphatidic acid; lauryl-glycerophosphatidic acid; oleoyl-glycerophosphatidic acid; n-alkyl-phosphorylglycerol; lauryl-phosphorylglycerol; oleoyl-phosphorylglycerol; n-alkyl-phosphorylserine; lauryl-phosphorylserine; oleoyl-phosphorylserine; n-tetradecyl-glycerophosphatidic acid; n-tetradecyl-phosphorylglycerol; n-tetradecyl-phosphorylserine; palmitoeloyl-glycerophosphatidic acid; palmitoeloyl-phosphorylglycerol; palmitoeloyl-phosphorylserine; elaidoyl-glycerophosphatidic acid; elaidoyl-phosphorylglycerol; elaidoyl-phosphorylserine; vaccenyl-glycerophosphatidic acid; vaccenyl-phosphorylglycerol; or vaccenyl-phosphorylserine.

78. The composition of claim 2, wherein the surface of the vesicle is positively charged and wherein the macromolecule is positively charged.

79. The composition of claim 30, wherein the surface of the vesicle is positively charged and wherein the macromolecule is positively charged.
